(19) United States
(12) Patent Application Publication
(10) Pub. No.: US 2025/0346604 A1
(43) Pub. Date: Nov. 13, 2025

(54) TRIAZOLOPYRIDAZINE COMPOUNDS USEFUL AS RAC1 INHIBITORS

(71) Applicant: Revere Pharmaceuticals, Waltham, MA (US)

(72) Inventors: Erik T. Goka, Waltham, MA (US); I. Robert Silverman, Arlington, MA (US); Alan Bruce Cooper, West Caldwell, NJ (US)

(21) Appl. No.: 19/188,531

(22) Filed: Apr. 24, 2025

Related U.S. Application Data
(63) Continuation of application No. PCT/US2023/035760, filed on Oct. 24, 2023.
(60) Provisional application No. 63/461,434, filed on Apr. 24, 2023.

(30) Foreign Application Priority Data
Oct. 25, 2022 (WO) PCT/US2022/047717

(51) Int. Cl. C07D 487/04; A61K 31/5025; C07B 59/00

(52) U.S. Cl. CPC C07D 487/04; A61K 31/5025; C07B 59/002

(57) ABSTRACT

Disclosed is a pharmaceutical composition comprising a pharmaceutically acceptable excipient, carrier or diluent and a compound of Formula I:

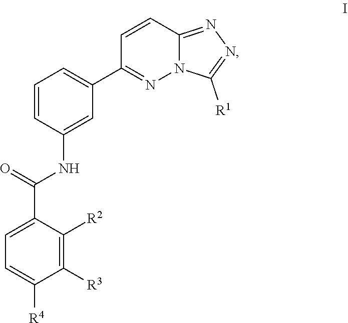

or a pharmaceutically acceptable salt thereof. The variables are defined herein. Also disclosed is a method of treating cancer with the pharmaceutical formulation.

### RELATED APPLICATION

This application is a continuation of International Application No. PCT/US2023/035760, filed Oct. 24, 2023, which claims the benefit of priority to PCT/US2022/047717, filed Oct. 25, 2022 and the benefit of U.S. Provisional Application Ser. No. 63/461,434, filed Apr. 24, 2023. The entire contents of these applications are incorporated herein by reference.

### BACKGROUND

The Rho GTPases Rac (Ras-related C3 botulinum toxin substrate) and Cdc42 (cell division control protein 42 homolog) regulate many cell functions, including cell polarity, migration, and cell cycle progression. The Rho family of GTPases in humans consists of 20 different members, and aberrant behavior in their regulatory activity has been implicated in cancer and other diseases. More than 70 guanine nucleotide exchange factors (GEFs) are known, which specifically activate one or more of the GTPases. In turn, the activated GTPases can specifically interact with over 60 downstream effectors. Dysregulation of one or more cellular processes can lead to release of malignant cells from their original locations, which subsequently can establish themselves in pre-metastatic niches in, for example, bone or lungs. It has been found that members of the Rho GTPase family, including Rac, Cdc42 and Rho, play key signaling roles in these processes.

This application claims the benefit of International Application Serial No. PCT/US2022/047717, filed Oct. 25, 2022 and the benefit of U.S. Provisional Application Ser. No. 63/461,434, filed Apr. 24, 2023. The entire teachings of both applications are incorporated herein by reference.

Rho GTPases regulate migration and invasion, cytoskeletal organization, transcriptional regulation, cell cycle progression, apoptosis, vesicle trafficking, and cell-to-cell and cell-to-extracellular matrix adhesions. The Rho GTPases Rac and Cdc42 are potent inducers of actin polymerization and extension of actin structures at the leading edge of motile cells. In addition, Cdc42 plays a critical role in cell polarity, and thus, promotes directed and persistent migration.

Hyperactive Rac and Cdc42 are associated with increased cancer cell survival, proliferation, and invasion, as well as Ras and other oncogene-mediated transformation. Furthermore, oncogenic cell surface receptors, such as tyrosine kinase, cytokine, and G protein coupled receptors, activate Rac and Cdc42 via regulation of their upstream effector GEFs.

Despite the recognized role of Rac1 in promoting tumor progression, there are no approved drugs that target this signaling protein. Although a handful of Rac1 inhibitors have been reported, these inhibitors have not been suitable for clinical development due to low potency or poor drug properties.

NSC23766 was identified as a small molecule that binds to a putative binding pocket in the surface groove of RacI that interacts with the Rac-specific GEFs Trio and Tiam1.

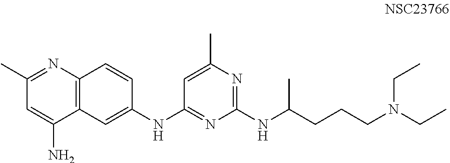 [SMILES: CCN(CC)CCCC(C)Nc1nc(C)cc(Nc2ccc3nc(C)cc(N)c3c2)n1]

NSC23766 has been shown to inhibit the anchorage-independent growth and invasion of human prostate cancer PC-3 cells as well as Rac activation and Rac-dependent aggregation of platelets stimulated by thrombin. It also inhibits Rac1 and Rac2 activities of hematopoietic stem/progenitor cells and migration from mouse bone marrow to peripheral blood. NSC23766 has also been shown to inhibit invasion of chronic myelogenous leukemia cells in vitro and in vivo in a mouse model. However, NSC23766 is a relatively weak Rac inhibitor, with a high IC₅₀ of 50-100 μM in fibroblasts. The weak activity of NSC23766 limits its potential use as a therapeutic agent.

U.S. Pat. No. 8,884,006 discloses a derivative of NSC23766, EHop-016, that is a more potent inhibitor of Rac1:

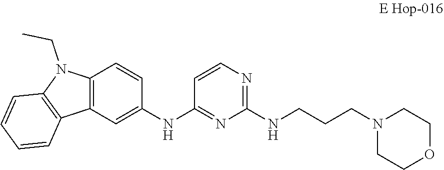 [SMILES: CCn1c2ccccc2c2cc(Nc3ccnc(NCCCN4CCOCC4)n3)ccc21]

EHop-016 is reported to be 100-fold more efficient than NSC23766 as an inhibitor of Rac activity. In MDA-MB-435 breast cancer cells, EHop-016 inhibits the association of the Rac-GEF Vav2 with a nucleotide-free Rac I (G15A), which hasa high affinity for activated GEFs. EHop-016 does not affect the association of the Rac-GEF Tiam-1with Rac1 (G15A) at similar concentrations. EHop-016 also inhibits the Rac activity of MDA-MB-231 metastatic breast cancer cells and reduces Rac-directed lamellipodia formation in both cell lines. Despite its improved potency, EHop-016 does not have a favorable in vivo pharmacokinetic profile, with low systemic exposure after oral administration in mice. Humphries-Bickley et al. J Chromatography B (2015), Volume 981-982, 19-26.

U.S. Pat. No. 1,047,235 and Molecular Cancer Therapeutics (2019), 18(5), 957-968 describes GY S32661:

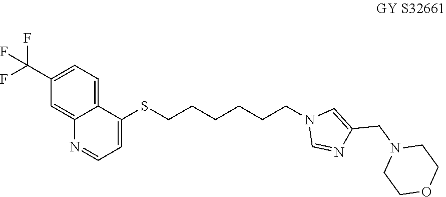 [SMILES: FC(F)(F)c1ccc2c(SCCCCCCn3cnc(CN4CCOCC4)c3)ccnc2c1]

GY S32661 shows very good activity against animal models of estrogen positive and HER2 positive breast cancer, prostate cancer, melanoma and colorectal cancer as a single agent and in combination with standard of care.

While Rac and Cdc42 GTPases are hyperactive or overexpressed in many types of cancer, there are no drugs for these important targets. As there is a continuing need for new therapeutic agents to treat cancer and other hyperproliferative diseases, it is desirable to have new inhibitors of Rac and/or Cdc42 with improved activity and pharmacokinetic properties.

### SUMMARY OF THE INVENTION

It has now been found that certain triazolopyridazine compounds disclosed herein have potent activity against wild type Rac1 and certain genomic variants thereof such as Rac1b and Rac1 P29S. A number of these compounds are significantly more potent than the aforementioned prior art compounds with an IC₅₀ inhibitory concentration versus wild type Rac1 below 1.0 μM in an AlphaL isa assay (see the section entitled “In Vitro Assays” below subsection 2 and Tables 1-6). By comparison, the reference standard Ehop-016 has an IC₅₀ of about 10 μM in the same assay. Additionally, certain of the disclosed triazolopyridazine compounds are selective for Rac1 over a panel of over 90 kinases (KinaseScan by Eurofins Discovery). Based on this discovery, pharmaceutical compositions comprising the disclosed Rac1 inhibitors and methods of treating cancer with the disclosed Rac1 inhibitors are described herein.

One embodiment of the invention is a compound (Rac1 inhibitor) of Formula I:

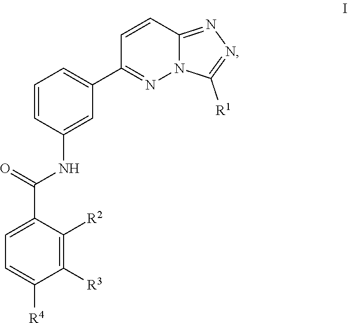

or a pharmaceutically acceptable salt thereof, wherein:
   R¹ is a 5-membered heteroaryl selected from furanyl, oxazolyl, isoxazolyl, pyrrolyl, pyrazolyl orthiazolyl wherein R¹ is optionally substituted by C₁₋₃ alkyl, C₁₋₃ hydroxyalkyl, C₁₋₃ alkyl carbonyl, C₁₋₃ haloalkyl or C₁₋₃ haloalkylcarbonyl;
   R² is H or F;
   R³ is halo, C₁₋₅ alkyl, —S(C₁₋₄alkyl), C₁₋₅ haloalkyl, C₁₋₄ alkoxy, —CO(C₁₋₄ alkyl), —CONH(C₁₋₄ alkyl), —CON(C₁₋₄alkyl)₂, —CO₂(C₁₋₄alkyl), —NH(C₁₋₄alkyl), —N(C₁₋₄ alkyl)₂, or C₁₋₄ haloalkoxy; and
   R⁴ is H or F.

Another embodiment of the invention is a pharmaceutical composition comprising: i) a pharmaceutically acceptable carrier, diluent or excipient; and ii) and a compound disclosed herein (Rac1 inhibitor disclosed herein) or a pharmaceutically acceptable salt thereof.

Another embodiment of the invention is a method of treating cancer in a patient comprising administering to the patient an effective amount of a compound disclosed herein (Rac1 inhibitor disclosed herein) or a pharmaceutically acceptable salt thereof; or pharmaceutical composition disclosed herein. Alternatively, the invention is a compound disclosed herein (Rac1 inhibitor disclosed herein) or a pharmaceutically acceptable salt thereof; or a pharmaceutical composition disclosed herein for treating cancer in a patient. In another alternative, the invention is the use of a compound disclosed herein (Rac1 inhibitor disclosed herein) or a pharmaceutically acceptable salt thereof; or a pharmaceutical composition disclosed herein for the manufacture of a medicament for treating cancer in a patient.

Another embodiment of the invention is a disclosed Rac1 inhibitor in which one or more hydrogen atoms are replaced by deuterium. Also included are pharmaceutical compositions comprising the deuterated Rac1 inhibitors and methods of treating cancer in a patient by administering to the patient an effective amount of the deuterated Rac1 inhibitors or pharmaceutical compositions comprising the same.

Another embodiment of the invention is a Rac1 inhibitor disclosed in Tables 1-3, or a pharmaceutically acceptable salt thereof.

### DETAILED DESCRIPTION OF THE INVENTION

Disclosed herein are Rac1 inhibitors which can be used in the treatment of a variety of cancers and pharmaceutical compositions comprising the same and a pharmaceutically acceptable excipient, carrier or diluent.

The Rac1 inhibitors disclosed herein are represented by Formula I above or a pharmaceutically acceptable salt thereof.

In a first aspect, the Rac1 inhibitor is represented by Formula I or a pharmaceutically acceptable salt thereof, wherein R¹ is a substituted furanyl or thiazolyl, and the remainder of the variables are as described for Formula I. Particular examples of R³ in Formula I include fluoro, chloro, bromo, ethyl, isobutyl, S(isopropyl), methoxy, and ethoxy.

In a second aspect, the Rac1 inhibitor is represented by Formula I or a pharmaceutically acceptable salt thereof, wherein R¹ is a substituted furanyl (e.g., substituted with C₁₋₃alkyl, C₁₋₃ hydroxyalkyl, C₁₋₃alkylcarbonyl, C₁₋₃haloalkyl or C₁₋₃ haloalkylcarbonyl), and the remainder of the variables are as described for Formula I.

In a third aspect, the Rac1 inhibitor is represented by Formula I or a pharmaceutically acceptable salt thereof, wherein R¹ is 5-methyl-furan-2-yl and the remainder of the variables are as described for Formula I.

In a fourth aspect, the Rac1 inhibitor is represented by Formula I or a pharmaceutically acceptable salt thereof, wherein R 1 is 5-methyl-furan-2-yl, R² is hydrogen and the remainder of the variables are as described for Formula I.

In a fifth aspect, the Rac1 inhibitor is represented by Formula I or a pharmaceutically acceptable salt thereof, wherein R¹ is a substituted furanyl, R² is hydrogen and the remainder of the variables are as described for Formula I.

In a sixth aspect, the Rac1 inhibitor is represented by Formula I or a pharmaceutically acceptable salt thereof, wherein R 1 is a substituted furanyl; R² is hydrogen; R³ is halo; and R⁴ is as described for Formula I.

In a seventh aspect, the Rac1 inhibitor is represented by Formula I or a pharmaceutically acceptable salt thereof, wherein R¹ is 5-methyl-furan-2-yl; R² is hydrogen; R³ is halo; and R⁴ is as described for Formula I.

In an eighth aspect, the Rac1 inhibitor is represented by Formula I or a pharmaceutically acceptable salt thereof, wherein R¹ is 5-methyl-furan-2-yl; R² is hydrogen; R³ is chloro; and R⁴ is as described for Formula I.

In a ninth aspect, the Rac1 inhibitor is represented by Formula I or a pharmaceutically acceptable salt thereof, wherein R¹ is thiazolyl (e.g., thiazol-5-yl) optionally substituted by C₁₋₃ alkyl, C₁₋₃ hydroxyalkyl, C₁₋₃alkylcarbonyl, C₁₋₃haloalkyl or C₁₋₃ haloalkylcarbonyl, and the remainder of the variables are as described for Formula I.

In a tenth aspect, the Rac1 inhibitor is represented by Formula I or a pharmaceutically acceptable salt thereof, wherein R¹ is thiazolyl (e.g., thiazol-5-yl), and the remainder of the variables are as described for Formula I.

In an eleventh aspect, the Rac1 inhibitor is represented by Formula I or a pharmaceutically acceptable salt thereof, wherein R² is H and R³ is hydrogen, halo, C₁₋₅ alkyl, or C₁₋₄ alkoxy; and the remainder of the variables are as described for Formula I or the ninth or tenth aspects. Alternatively, R² is H and R³ is hydrogen, methoxy, chloro or fluoro. In another alternative, R² is H and R³ is hydrogen or methoxy.

In a twelfth aspect, the Rac1 inhibitor is represented by Formula I or a pharmaceutically acceptable salt thereof, wherein R² is H and R³ is hydrogen, halo, C₁₋₅alkyl, or C₁₋₄ alkoxy; and the remainder of the variables are as described for Formula I, the second aspect or the third aspect. Alternatively, R² is H and R³ is hydrogen, chloro or fluoro.

In a thirteenth aspect, the Rac1 inhibitor is any one of the compounds listed in Tables 1-3 below, or a pharmaceutically acceptable salt thereof.

Particular examples of R¹ in Formula I include the following:

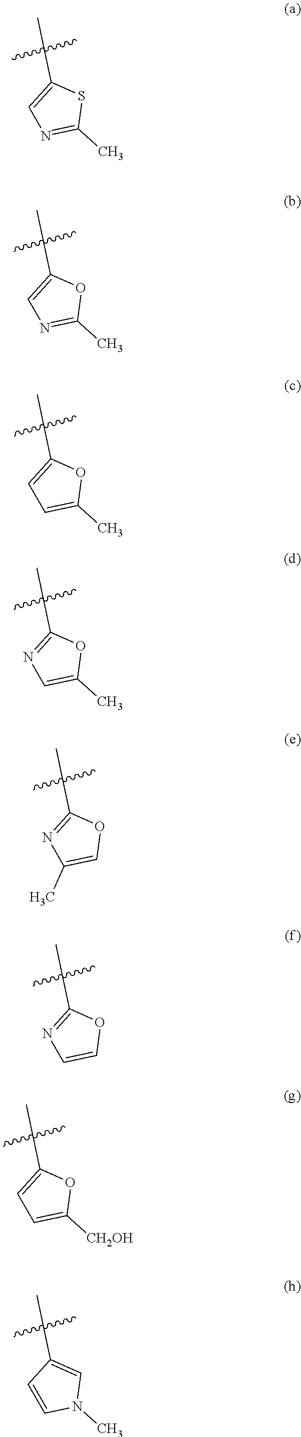

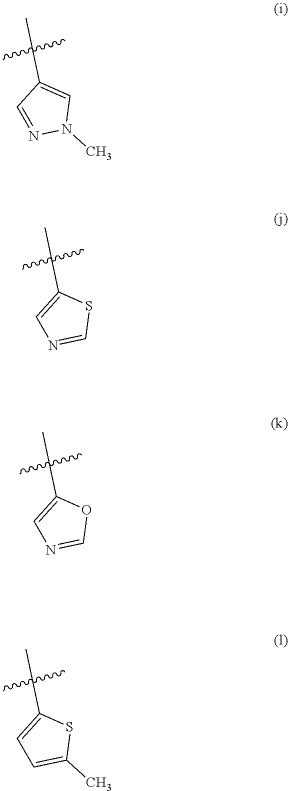

Another embodiment of the invention is a pharmaceutical composition comprising a pharmaceutically acceptable excipient, carrier or diluent and a compound of Formula II:

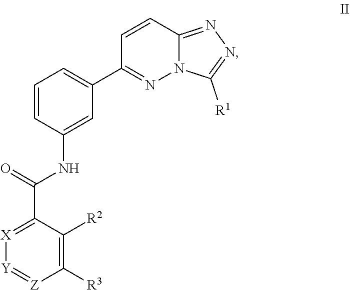

wherein R¹ and R² are as described above for Formula I;
one of X, Y and Z is nitrogen and each of the others are CH;
when Z is CH, R³ is halo, C₁₋₅ alkyl, —S(C₁₋₄ alkyl), C₁₋₅ haloalkyl, C₁₋₄ alkoxy, —CO(C₁₋₄ alkyl), —CONH(C₁₋₄alkyl), —CON(C₁₋₄alkyl)₂, —CO₂(C₁₋₄alkyl), —NH(C₁₋₄alkyl), —N(C₁₋₄ alkyl)₂, or C₁₋₄ haloalkoxy; and
when Z is N, R³ is C₁₋₅ alkyl, —S(C₁₋₄alkyl), C₁₋₅ haloalkyl, —CO(C₁₋₄alkyl), —CONH(C₁₋₄ alkyl), —CON(C₁₋₄alkyl)₂, or —CO₂(C₁₋₄alkyl).

Another embodiment of the invention is a pharmaceutical composition comprising a pharmaceutically acceptable excipient, carrier or diluent and a compound of Formula IIIa or IIIb:

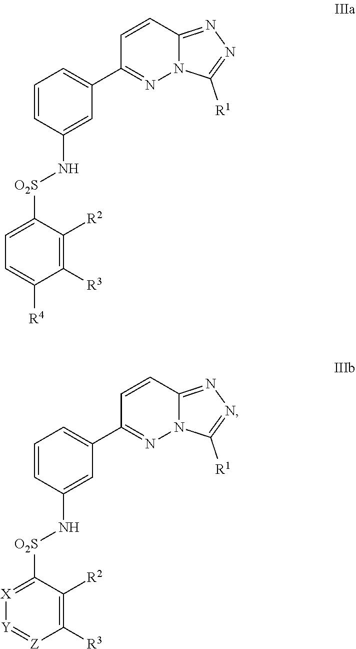

wherein R¹, R², R³, R⁴, X, Y and Z are as described above.

Exemplary compounds of the invention are described in Tables 1-3 and in the Exemplification section below. Pharmaceutically acceptable salts of these compounds are also included in the invention as well as the neutral form.

TABLE 1
Examples of Compounds of Formula I-A (R¹ is
5-methyl-2-furanyl; where R² = H)

| | |
|---|---|
| | I-A |
| 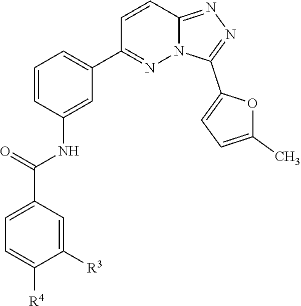 | |

| Compound | | | |
|---|---|---|---|
| No. | R³ | R⁴ | Activity |
| 100 | OCH₃ | H | ††† |
| 101 | F | H | ††† |
| 102 | Cl | H | ††† |
| 103 | Cl | F | ††† |
| 104 | Br | H | ††† |
| 105 | F | H | ††† |
| 106 | F | F | ††† |
| 107 | SCH₃ | F | ††† |
| 108 | SCH₂CH₃ | H | ††† |
| 109 | SCH(CH₃)₂ | H | ††† |
| 110 | CH₃ | H | ††† |
| 111 | CH₂CH₃ | H | ††† |
| 112 | SCH(CH₃)₂ | F | ††† |
| 113 | CH₃ | F | ††† |
| 114 | CH₂CH₃ | F | ††† |
| 115 | Br | F | ††† |
| 116 | 5-oxazolyl | CH₃ | ††† |
| 117 | CH₂CH₂CH(CH₃)₂ | F | ††† |
| 118 | 3-S(tBu) | H | ††† |

TABLE 2
Examples of Compounds of Formula 1-B
(R¹ is thiazol-5-yl; where R² is H):

| | |
|---|---|
| | I-B |
| 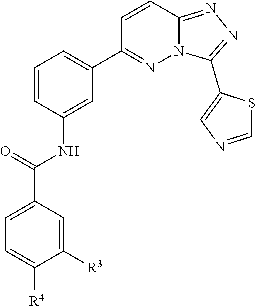 | |

| Compound | | | |
|---|---|---|---|
| No. | R³ | R⁴ | IC₅₀ (uM) |
| 200 | OCH₃ | H | ††† |
| 201 | H | F | ††† |
| 202 | Cl | H | †† |
| 203 | Cl | F | †† |
| 204 | CH₂CH₃ | H | NA |
| 205 | CH₂CH₃ | F | NA |
| 206 | SCH₂CH₃ | H | NA |

TABLE 3
Examples of other Compounds of Formula I (where R² is H):

| | | | | I |
|---|---|---|---|---|
| 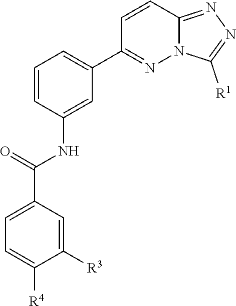 | | | | |
| (R² = H) | | | | |
| Compound | | | | IC₅₀ |
| No. | R¹ | R³ | R⁴ | (uM) |
| 300 | 5-hydroxymethyl-furan-2-yl (g) | OCH₃ | F | ††† |
| 301 | 5-formyl-furan-2-yl | H | F | ††† |
| 302 | 5-carboethoxy-furan-2-yl | H | F | NA |
| 303 | 5-methyl-thien-2-yl | Cl | H | †† |
| 304 | 5-methyl-thien-2-yl | H | F | ††† |

TABLE 4
Comparator Compounds

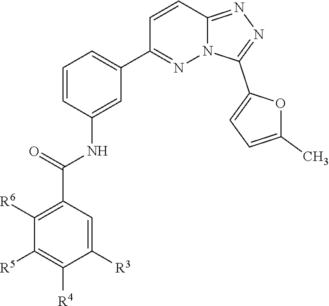

| Compound | | | | | |
|---|---|---|---|---|---|
| No. | R³ | R⁴ | R⁵ | R⁶ | IC₅₀ (uM) |
| Comparator | OCH₃ | OCH₃ | H | H | †† |
| 1 | | | | | |
| Comparator | OCH₃ | OCH₃ | OCH₃ | H | † |
| 2 | | | | | |
| Comparator | OCH₃ | H | H | OCH₃ | † |
| 3 | | | | | |

Activity values reported in Tables 1-3 were obtained from the Rac Activation AlphaScreen Assay (RacI AS) assay described in subsection 2 under the section entitled “In Vitro Assays”. “†” represents an IC₅₀ of greater than 10 uM; “††” represents an IC₅₀ of greater than 1 uM and less than or equal to 10 uM; and “†††” represents an IC₅₀ of less than or equal to 1 uM. “NT” means not tested.

Another embodiment of the invention is a disclosed Rac1 inhibitor in which one or more hydrogen atoms are replaced by deuterium. When a hydrogen atom is replaced by deuterium at a particular position, the position is understood to have deuterium at an abundance that is at least 3000 times greater than the natural abundance of deuterium, which is 0.015% (i.e., at least 45% incorporation of deuterium). Alternatively, the deuterium incorporation is at least 52.5% at each designated position, at least 60% at each designated position, at least 67.5% at each designated position, at least 75% at each designated position, at least 82.5% at each designated position, at least 90% at each designated position, at least 95% at each designated position, at least 97% at each designated position, at least 99% at each designated position, or at least 99.5% at each designated position.

In one aspect, the substituent on R¹ and/or the R³ group of the disclosed Rac1 inhibitors are deuterated when R¹ has an alkyl substituent or a substituent with an alkyl moiety and/or when R³ is alkyl, alkoxy or has an alkyl or alkoxy moiety. In another aspect, the alkyl substituent on R¹ and/or the alkyl or alkoxy group represented by R³ are perdeuterated, i.e., all of the hydrogen atoms are replaced with deuterium. In another aspect, the deuterated groups on R¹ or represented by R³ are —CD₃ (for R¹) and/or —CD₃ or —OCD₃ (for R³), and in another aspect, these groups are perdeuterated.

When a position is designated specifically as “H” or “hydrogen”, the position is understood to have hydrogen at its natural abundance isotopic composition. When a position is designated specifically as “D” or “deuterium”, the position is understood to be enriched in deuterium, as described above. When there is no specific designation as to whether a position has hydrogen or deuterium, it is understood that the position has hydrogen at natural abundance. For example, the term “methyl”, unless there is a specific designation to the contrary, is understood to mean —CH₃ with all three hydrogen atoms present at natural abundance, and the term “phenyl”, unless there is a specific designation to the contrary, means all five hydrogen atoms to be present at natural abundance.

An example of a deuterated compound of the invention is shown below as Formula IV:

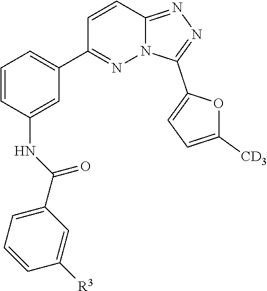

Pharmaceutically acceptable salts of the compound of Formula IV are included. R³ is hydrogen, halo, C₁₋₅ alkyl, or C₁₋₄ alkoxy. Alternatively, R³ is hydrogen, chloro or fluoro. In another alternative, R³ is chloro.

As used herein, “alkyl” refers to a fully saturated branched or unbranched hydrocarbon moiety. Unless otherwise specified, an alkyl comprises 1 to 4 carbon atoms. Representative examples of alkyl include, but are not limited to, methyl, ethyl, n-propyl, iso-propyl, n-butyl, sec-butyl, iso-butyl and tert-butyl.

“Halogen” or “halo” may be fluoro, chloro, bromo or iodo.

In many cases, the compounds of the present invention can form acid salts by virtue of the presence of basic nitrogen atom(s). As used herein, the terms “salt” or “salts” refers to an acid addition salt of a compound of the invention. “Salts” include in particular “pharmaceutically acceptable salts”. The term “pharmaceutically acceptable salts” refers to salts that retain the biological effectiveness and properties of the compounds of this invention and, which typically are not biologically or otherwise undesirable.

Pharmaceutically acceptable acid addition salts can be formed with inorganic acids and organic acids, e.g., tosylate, methanesulfonate, acetate, citrate, malonate, tartarate, succinate, benzoate, hydrochloride, sulfate, nitrate, bicarbonate, phosphate and carbonate salts. Lists of additional suitable salts can be found, e.g., in REMINGTON'S PHARMACEUTICAL SCIENCES, 20th ed., Mack Publishing Company, Easton, Pa., (1985); and in HANDBOOK OF PHARMACEUTICAL SALTS: PROPERTIES, SELECTION, AND USE, by Stahl and Wermuth (Wiley-VCH, Weinheim, Germany, 2002).

A s used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, REMINGTON'S PHARMACEUTICAL SCIENCES, 18th Ed. Mack Printing Company, 1990, pp. 1289-1329). Except insofar as any conventional carrier is incompatible with the active ingredient, its use in the therapeutic or pharmaceutical compositions is contemplated.

The term “effective amount” (used interchangeably with “therapeutically effective amount”) of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or reduce the likelihood or delay reoccurrence of a disease, etc. In one non-limiting embodiment, the term “a therapeutically effective amount” refers to the amount of the compound of the present invention that, when administered to a subject, is effective to (1) at least partially alleviate, inhibit and/or ameliorate a condition, or a disorder or a disease (i) mediated by hyperactivation of Rac1 or (ii) associated with overexpression of Rac1, or (iii) characterized by activity (normal or abnormal) of Rac1.

In another non-limiting embodiment, the term “a therapeutically effective amount” or “an effective amount” refers to the amount of the compound of the present invention that, when administered to a cell, or a tissue, or a non-cellular biological material, or a medium, is effective to at least partially reduce or inhibit the activity of Rac1, or at least partially reduce or inhibit the expression of Rac1.

As used herein, the term “subject” refers to an animal. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In specific embodiments, the subject is a human.

As used herein, the term “inhibit”, “inhibition” or “inhibiting” refers to the reduction or suppression of a given condition, activity, effect, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to delaying the progression of the disease or disorder. In another embodiment, “treat”, “treating” or “treatment” refers to reducing the likelihood or delaying reoccurrence of the disease or disorder after it has gone into remission.

The pharmaceutical composition or combination of the present invention can be in unit dosage of about 1-2000 mg of active ingredient(s) for a subject of about 50-70 kg, of active ingredients. The therapeutically effective dosage of a compound, the pharmaceutical composition, or the combinations thereof, is dependent on the species of the subject, the body weight, age and individual condition, the disorder or disease being treated and the severity thereof. A physician, clinician or veterinarian of ordinary skill can readily determine the effective amount of each of the active ingredients necessary to prevent, treat or inhibit the progress of the disorder or disease.

As indicated above, a further embodiment of the invention relates to a pharmaceutical composition comprising at least one compound of the invention and a pharmaceutically acceptable diluent, excipient, or carrier.

The compounds of the invention are typically administered with suitable pharmaceutical diluents, excipients, or carriers (collectively referred to herein as pharmaceutically acceptable carriers). Suitable pharmaceutical diluents, excipients, and carriers include, but are not limited to, lubricants, solvents, binders, and stabilizers that are suitably selected with respect to the intended form of administration including solid and liquid forms, such as capsules, tablets, gels, solutions, syrups, suspensions, powders, aerosols, ointments, and the like.

Diluents that may be used in the compositions of the invention include but are not limited to dicalcium phosphate, calcium sulfate, lactose, cellulose, kaolin, mannitol, sodium chloride, dry starch and powdered sugar. The binders that may be used in the compositions of the invention include but are not limited to starch and gelatin. Additionally, fillers such as sucrose, glucose, dextrose and lactose may also be used.

Natural and synthetic gums that may be used in the compositions of the invention include but are not limited to sodium alginate, ghatti gum, carboxymethyl cellulose, methyl cellulose, polyvinyl pyrrolidone and veegum. Excipients that may be used in the compositions of the invention include but are not limited to microcrystalline cellulose, calcium sulfate, dicalcium phosphate, starch, magnesium stearate, lactose, and sucrose. Stabilizers that may be used in the compositions of the invention include but are not limited to polysaccharides such as acacia, agar, alginic acid, guar gum and tragacanth, amphotsics such as gelatin and synthetic and semi-synthetic polymers such as carbomer resins, cellulose ethers and carboxymethyl chitin.

Solvents that may be used in the composition of the invention include but are not limited to Ringers solution, water, distilled water, dimethyl sulfoxide to 50% in water, propylene glycol (neat or in water), phosphate buffered saline, balanced salt solution, glycol and other conventional fluids.

The dosages and dosage regimen in which the compounds of the invention are administered will vary according to the dosage form, mode of administration, the condition being treated and particulars of the patient being treated. Accordingly, optimal therapeutic concentrations will be best determined at the time and place through routine experimentation.

The compounds according to the invention can also be used enterally. Orally, the compounds according to the invention are suitably administered at the rate of 10 μg to 300 mg per day per kg of body weight. The required dose can be administered in one or more portions. For oral administration, suitable forms are, for example, capsules, tablets, gels, aerosols, pills, dragees, syrups, suspensions, emulsions, solutions, powders and granules. A preferred method of administration consists of using a suitable form containing from 0.01 mg to about 500 mg of active substance.

The compounds according to the invention can also be administered parenterally in the form of solutions or suspensions for intravenous, subcutaneous or intramuscular perfusions or injections. In that case, the compounds according to the invention are generally administered at the rate of about 10 μg to 10 mg per day per kg of body weight. A preferred method of administration consists of using solutions or suspensions containing approximately from 0.01 mg to 1 mg of active substance per ml.

The compounds may be administered according to various routes, typically by oral route or by injection, such as local or systemic injection(s). Intratumoral injections are preferred for treating existing cancers. However, other administration routes may be used as well, such as intramuscular, intravenous, intradermic, subcutaneous, etc. Furthermore, repeated injections may be performed, if needed, although it is believed that a limited number of injections will be needed in view of the efficacy of the compounds.

The compounds of the invention can be used in a substantially similar manner to other known anti-tumor agents for treating (both chemopreventively and therapeutically) various tumors. The dose to be administered, whether a single dose, multiple dose, or a daily dose, will vary with the particular compound employed because of the varying potency of the compound, the chosen route of administration, the size of the recipient, the type of disease, and the nature of the patient's condition. The dosage to be administered is not subject to definite bounds, but it will usually be an effective amount, or the equivalent on a molar basis of the pharmacologically active free form produced from a dosage formulation upon the metabolic release of the active drug to achieve its desired pharmacological and physiological effects. An oncologist skilled in the art of cancer treatment or a doctor skilled in the art in treating kidney or heart disease will be able to ascertain, without undue experimentation, appropriate protocols for the effective administration of the compounds of this present invention.

The compounds of the invention may also be administered in combination with other known therapies. For example, the compounds of the invention can be administered in combination with other known chemotherapy drugs. When co-administered with one or more other therapies, the compounds of the invention can be administered either simultaneously with the other treatment(s), or sequentially. If administered sequentially, the attending physician will decide on the appropriate sequence of administering the compounds of the invention in combination with the other therapy.

Scheme 1 below shows the preparation of compounds of Formula I, exemplified for compounds where R¹ is a 2-furanyl.

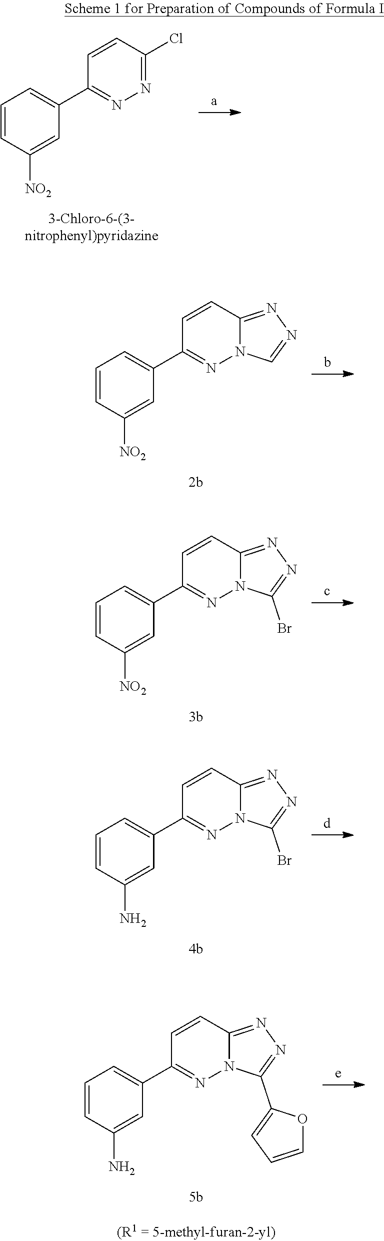 [SMILES: O=[N+]([O-])c1cccc(-c2ccc(Cl)nn2)c1 ; O=[N+]([O-])c1cccc(-c2ccc3nncn3n2)c1 ; O=[N+]([O-])c1cccc(-c2ccc3nnc(Br)n3n2)c1 ; Nc1cccc(-c2ccc3nnc(Br)n3n2)c1 ; Nc1cccc(-c2ccc3nnc(-c4ccco4)n3n2)c1]

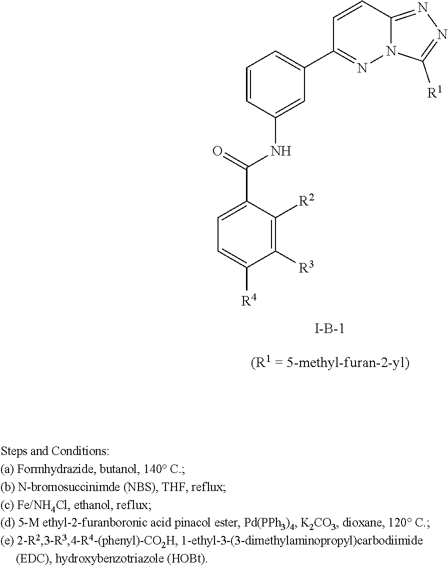

Compounds of Formula I were prepared according to Scheme 1. The known 3-chloro-6-(3-nitrophenyl)pyridazine, which is commercially available, was condensed with formhydrazide according to step (a) to provide the triazolopyridazine 2b. Bromination with NBS gave the bromo intermediate 3b, which was reduced to the amine 4b as shown in step (c). Suzuki-type coupling with 5-methyl-2-furanboronic acid pinacol ester provided 5b. Intermediate 5b was generally useful for preparing compounds of Formula I-A where R¹ is 5-methyl-furan-2-yl employing standard amide coupling conditions as shown in step (e). Scheme 1 may be readily adapted to prepare other compounds of Formula I having other R¹ substituents that are 5-6 membered heteroaryl rings by using the boronic ester of the desired R¹ substituent.

Standard coupling reactions were useful in preparing a number of compounds of the invention having various R¹ groups, examples of which are shown in Table 5 below.

TABLE 5
Examples of Coupling Reactions for Preparing Compounds of Formula I Having
Various R¹ Groups

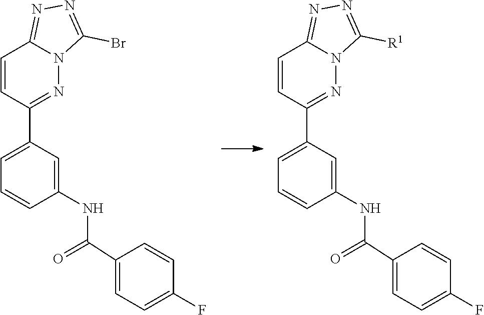

| Compound | | | |
|---|---|---|---|
| No. | Coupling Partner | Reaction Conditions | R¹ |
| 95 | 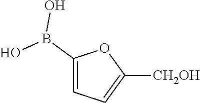 | PdCl₂(dppf), K₂CO₃, dioxane, 110° C., 12 h | 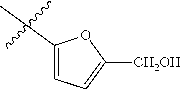 [SMILES: *c1ccc(CO)o1] |
| 99 | 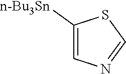 | PdCl₂(dppf), dioxane, 140° C. | 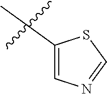 [SMILES: *c1cncs1] |
| 112 | 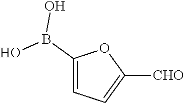 | PdCl₂(PPh₃)₂, CsCO₃, dioxane, 110° C., 12 h | 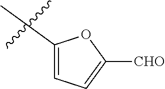 [SMILES: *c1ccc(C=O)o1] |
| 113 | 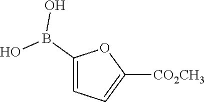 [SMILES: COC(=O)c1ccc(B(O)O)o1] | PdCl₂(PPh₃)₂, CsCO₃, dioxane, 110° C., 12 h | 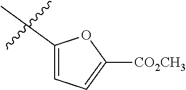 [SMILES: *c1ccc(C(=O)OC)o1] |

Another synthetic approach for preparing compounds of the invention is shown below in Scheme 2 for exemplary compounds of Formula I where R¹ is a 5-methyl-2-furanyl.

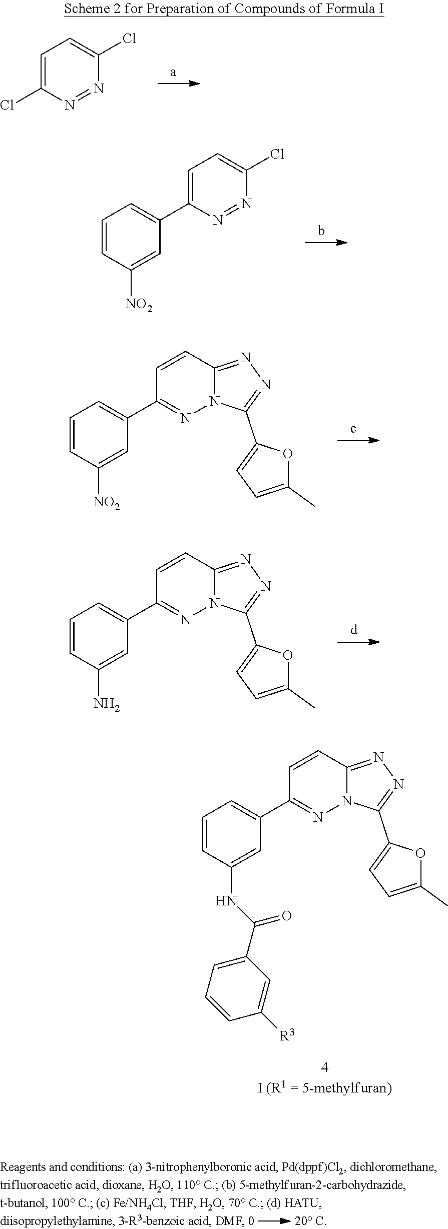

Certain deuterated compounds of this invention represented by Formula IV can be prepared by replacing the hydrazide in step (b) with the appropriate deuterated analog. For example, deuterated compounds with a 5-methylfuranyl group may be prepared by replacing 5-methylfuran-2-carbohydrazide in step (b) with the following deuterated analog:

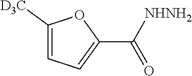 [SMILES: [2H]C([2H])([2H])c1ccc(C(=O)NN)o1]

This deuterated analog can be prepared according to Scheme 3 below:

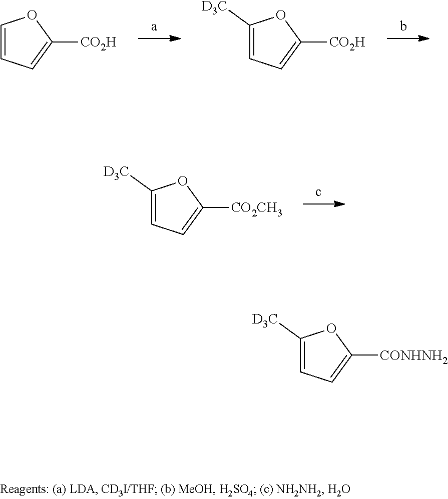

Another embodiment of the invention are the reaction products and salts thereof of step b and c in Scheme 2 above. Y et another embodiment of the invention are the three deuterated intermediates shown in Scheme 3 above and salts thereof.

Compound 119 (or a pharmaceutically acceptable salt thereof) is an example of a deuterated compound of the invention:

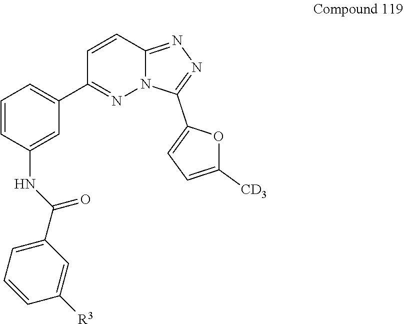

Compounds of Formula I where R¹ is optionally substituted furanyl or an optionally substituted oxazolyl, isoxazolyl, thiazolyl, furanyl, pyrrolyl, and pyrazolyl are new. As such, the invention is directed to a compound of formula I, wherein R¹ is optionally substituted furanyl or an optionally substituted oxazolyl, isoxazolyl, thiazolyl, furanyl, pyrrolyl, and pyrazolyl (suitable substituents include C₁₋₃ alkyl, C₁₋₃ hydroxyalkyl, C₁₋₃ alkylcarbonyl, C₁₋₃ haloalkyl or C₁₋₃ haloalkylcarbonyl), and the remainder of the variables are as described for Formula I and in aspect one through twelve.

The compounds of the invention are useful for treating cancers that show a dependence on Rac protein signaling for their growth and survival and, in particular, where the tumor progression is driven by dysregulation of Rac signaling. Specific examples include breast cancer, melanoma, ovarian cancer, head cancer, neck cancer, prostate cancer, colorectal cancer, pancreatic cancer, liver cancer, bladder cancer, non-Hodgkin's lymphoma, and leukemia (acute lymphoblastic leukemia, chronic myeloid leukemia, acute myeloid leukemia). Additionally, the compounds of the invention can be used to treat kidney disease and heart disease.

The invention also provides a method to treat a condition characterized by excessive or undesired levels of activity of Rac1, wherein the method comprises administering to a subject in need of such treatment an effective amount of a compound of Formula (I) or any subgenus thereof as described herein, or a pharmaceutical composition comprising such compound. The subject can be a mammal, and is preferably a human, and is typically a subject diagnosed with a condition associated with excessive activity of Rac1. Conditions treatable by the compounds and methods described herein include various forms of cancer that are responsive to Rac1 inhibitors, such as solid tumors, adenoma, bladder cancer, brain cancer, breast cancer, cervical cancer, colorectal cancer, colon cancer, epidermal carcinoma, follicular carcinoma, genitourinary cancers, glioblastoma, head and neck cancers, Hodgkin's disease, non-Hodgkin's lymphoma, hepatoma, kidney cancer, lung cancers such as small cell or non-small cell lung cancer, leukemias such as AML or CML, multiple myeloma, lymphoid disorders, skin cancers including melanoma, neuroblastoma, ovarian cancer, pancreatic cancer, prostate cancer, rectal cancer, sarcoma, testicular cancer, and thyroid cancer.

The compounds are especially indicated for use to treat melanoma, ovarian cancer, thyroid cancer, colon cancer, breast cancer, and prostate cancer. Indications of special interest for use of the compounds of the invention include cancers that express Rac1b, a splice variant of Rac1, and the mutant forms Rac1 P29S and Rac1 A 149V. Examples of breast cancer for use of the compounds include ER+ breast cancer that is resistant to ER targeted therapy and/or CD K 4/6 inhibitors; and HER2+ breast cancer that is resistant to HER² targeted therapies. Examples of prostate cancer for use of the compounds include castrate resistant prostate cancer (CRPC) that resistant to androgen receptor inhibitors. Examples of ovarian cancer for use of the compounds include ovarian cancer that is platinum resistant and for patients who are HRD negative.

In one aspect, the cancer is determined to exhibit a high expression level of Rac1 prior to treatment with a therapeutically effective amount of a Rac1 inhibitor. This determination can be made by routine diagnostic methods which obtain cancer cells from a patient. These methods include, but are not limited to, biopsy, blood tests, and other diagnostic methods which obtain samples of cancer cells such as tissue samples, circulating tumor cells, exosomes, or biomolecules characteristic of cancer such as circulating nucleic acids or proteins. The expression level of Rac1 in the cancer cells is then determined or inferred. Determining if the cancer exhibits a high expression level of Rac1 is by methodology known in the art, for example, by determining Rac1 expression levels in the isolated cancer cells by RNA sequencing (RNA-Seq), microarray, quantitative PCR, or NanoString™ gene expression panels, gene amplification by FISH, or Rac1 protein by immunohistochemistry, flow cytometry, immunocytochemistry or Western blot. See e.g., RT-qPCR analysis discussed below. In one embodiment, the methods disclosed herein further comprise a step of performing a biopsy of the patient's cancer prior to treatment and determining from the cancer cells isolated from the biopsy if the cancer (cancer cells) exhibits a high expression level of Rac1.

In one embodiment, the Rac1 expression level is determined for Rac1 wild type (Rac1 wt). In another embodiment, the Rac1 expression level is determined for a genomic variant of Rac1, such as the Rac1b splice variant and the Rac1 P29S mutant.

In another aspect, the invention is a method of treating a patient with a cancer comprising providing cancer cells from the cancer patient; determining the expression level of Rac1 in the cancer cells; and administering to the patient a therapeutically effective amount of a Rac1 inhibitor, if the patient's cancer (cancer cells) exhibits a high expression level of Rac1. In one embodiment, the method further comprises excluding the patient from administration of a Rac1 inhibitor if the patient's cancer (cancer cells) does not exhibit a high expression level of Rac1. The cancer cells used in the present invention can be obtained from a sample which is, but not limited to a sample of tissue, blood (including blood fractions), lymphatic fluid, sputum, feces, urine, bronchial lavage, or other body fluid.

In another aspect, provided herein is a method of selecting a patient who is likely to respond to treatment with a Rac1 inhibitor, said method comprising determining the expression level of Rac1 of a cancer of the patient, wherein the patient is likely to respond to treatment if the expression level of Rac1 by the cancer is high.

In another aspect, provided herein is a method of treating a patient with a cancer, comprising determining the expression level of Rac1 of the cancer and administering a therapeutically effective amount of a Rac1 inhibitor if the expression level of Rac1 by the cancer is high, and treating the patient with an anti-cancer therapy other than a Rac1 inhibitor if the patient's cancer does not exhibit a high expression level of Rac1.

In one aspect, the high expression level of Rac1 is characterized by an expression level falling within the top 50% of Rac1 expression levels of the cancer cells from the same cancer type in a random population of patients. The Rac1 expression level can be obtained from methods suitable for determining Rac1 expression levels, such as, e.g., expression levels derived from RNA-sequencing such as normalized read counts and TPM (Transcripts Per Million) or normalized cycle threshold (Ct) levels from RT-PCR measurements] for Rac1 robustly standardized (quantiles 2.5% and 97.5% set to −1 and +1, respectively).

As used herein “high expression” means an expression level falling within the top 5%, 10%, 15%, 20%, 25%, 30%, 40%, 45%, or 50% of the expression levels. “Top 50%”, for example, can be obtained by collecting expression levels of Rac1 from the cancer cells (e.g., from tissue samples) of a random population of subjects, e.g., at least 25 subjects, at least 50 subjects, at least 100 subjects, at least 500 subjects, at least 1000 subjects or the like, having the same cancers and then assessing whether the expression level of a new subject falls within the top 50% percentile.

In an alternative, “high expression” refers to a level of Rac1 in the cancer from the patient above a defined reference level of 25%, 30%, 40%, 50%, 60%, 70%, 80%, 85%, 90%, 95%, 100%, 150%, 200%, 250% or greater, determined by the methods described herein, as compared to the reference level.

“Reference level” refers to an average Rac1 expression level determined in cells of the same cell type as the cancer obtained from a population of healthy individuals without the cancer. In an alternative aspect, the reference level can be determined in non-cancerous cells of the same cell type as the cancer obtained from the patient.

In one aspect, the reference level can be obtained by determining the average normalized Rac1 expression [which can be obtained from methods suitable for determining Rac1 expression levels, such as, e.g., expression levels derived from RNA-sequencing such as normalized read counts and TPM (Transcripts Per Million) or normalized cycle threshold (Ct) levels from RT-PC R measurements] for Rac1 robustly standardized (quantiles 2.5% and 97.5% set to −1 and +1, respectively).

Some genomic variants of Rac1, such as Rac1 P29S, are only known to be expressed in cancerous cells. Thus, for such genomic variants, “high Rac1 expression” means a detectable level of such Rac1 genomic variant. Rac1 P29S is most prevalent in melanoma. Accordingly, one embodiment of this invention relates to a method of treating a cancer that expresses Rac1 P29S in a patient by administering to the patient an effective amount of a Rac1 P29S inhibitor of this invention. In one aspect, the cancer that expresses Rac1 P29S is melanoma.

Another embodiment of this invention provides a method of treating a cancer that is characterized by high expression of Rac1b in a patient by administering to the patient an effective amount of a Rac1b inhibitor. In one aspect, the cancer that highly expresses Rac1b is colorectal, non-small cell lung, small cell lung, breast, prostate, thyroid, hepatocellular, ovarian, esophageal, gastric, or pancreatic cancer. Expression of Rac1b can be determined by immunohistochemistry (IHC) staining of patient tissue sample using a Rac1b antibody using a scoring system that would be known to a trained pathologist. In a scoring system of 0-3, high Rac1 expression may be tissue having a score of at least 2 or at least 3.

Elevated levels of activated Rac1 or GTP-bound Rac1 are found in patients that have high expression levels of certain other proteins that activate Rac1 such as guanine nucleotide exchange factors (GEFs). At least 20 GEFs are involved in Rac1 activation. The GEFs Tiam1 and P-Rex1 are Rac1 specific. Accordingly, one embodiment of this invention relates to treating a cancer that is characterized by high expression levels of a GEF in a patient by administering to the patient an effective amount of a Rac1 inhibitor. In one aspect, the cancer is characterized by high expression levels of Tiam1 or P-Rex1. Methods for determining the expression levels of the GEF are available to those skilled in the art in manners that are analogous to those described above for determining the expression levels of Rac1.

Elevated levels of activated Rac1 or GTP-bound Rac1 are also found in patients that have low expression levels of proteins that are involved in the degradation of active Rac1. HACE-1 is an E3-ubiquitin ligase tumor suppressor that targets active Rac1 for degradation. Low levels of HACE-1 are associated with higher levels of active, GTP-bound Rac1. Accordingly, one embodiment of this invention relates to treating a cancer that is characterized by low expression levels of HACE-1 in a patient by administering to the patient an effective amount of a Rac1 inhibitor. Methods for determining the expression levels of HACE-1 are available to those skilled in the art in manners that are analogous to those described above for determining the expression levels of Rac1.

In one aspect, the low expression level of HACE-1 is characterized by an expression level falling within the bottom 50% of HACE-1 expression levels of the cancer cells from the same cancer type in a random population of patients. The HACE-1 expression level can be obtained from methods suitable for determining HACE-1 expression levels, such as, e.g., expression levels derived from RNA-sequencing such as normalized read counts and TPM (Transcripts Per Million) or normalized cycle threshold (Ct) levels from RT PCR measurements] for Rac1 robustly standardized (quantiles 2.5% and 97.5% set to −1 and +1, respectively).

As used herein “low expression” means an expression level falling within the bottom 5%, 10%, 15%, 20%, 25%, 30%, 40%, 45%, or 50% of the expression levels. “Bottom 50%”, for example, can be obtained by collecting expression levels of HACE-1 from the cancer cells (e.g., from tissue samples) of a random population of subjects, e.g., at least 25 subjects, at least 50 subjects, at least 100 subjects, at least 500 subjects, at least 1000 subjects or the like, having the same cancers and then assessing whether the expression level of a new subject falls within the bottom 50% percentile.

In an alternative, “low expression” refers to a level of HACE-1 in the cancer from the patient below a defined reference level by 25%, 30%, 40%, 50%, 60%, 70%, 80%, 85%, 90%, 95%, 100%, 150%, 200%, 250% or greater, determined by the methods described herein, as compared to the reference level.

Levels of HACE-1 may also be determined using known immunohistochemistry methods for the HACE-1 protein. See Da et al., Signal Transduction and Targeted Therapy 6, Article N umber 399 (2021) and Anglesio et al., Human Molecular Genetics 13(18), pp 2061-2074 (2004).

The invention is illustrated by the following examples, which are not intended to be limiting in any way.

### EXEMPLIFICATION

Abbreviations used in experimental procedures:
ACN Acetonitrile
aq. Aqueous
DCM Dichloromethane
DIPEA N,N-Diisopropylethylamine
DMF Dimethylformamide
DMSO Dimethyl sulfoxide
dppf 1,1′-Bis(diphenylphosphino)ferrocene
eq equivalent
Et₃N or TEA Triethylamine
EtOAc Ethyl acetate
EtOH Ethanol
FA Formic acid
g gram
h Hour
HATU Hexafluorophosphate Azabenzotriazole Tetramethyl Uronium
DIPEA Diisopropyl ethylamine
LCMS Liquid chromatograph mass spectrometry
LDA Lithium diisopropylamide
MeCN Acetonitrile
MeOH Methanol
mg milligram
min minute
ml or mL milliliters
mol mole
NBS N-bromosuccinimide
TEA Triethylamine
THF Tetrahydrofuran
TLC Thin layer chromatography
RBF Round bottom flask
RT Room temperature
tBuO H tert-butanol
T₃P Propane phosphoric acid anhydride

### Synthesis of Common Intermediate 11

### Step 1:

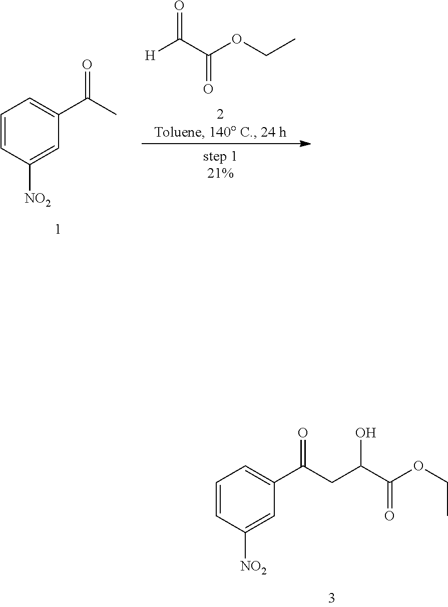

Ethyl 2-hydroxy-4-(3-nitrophenyl)-4-oxobutanoate (3): A mixture of compound 1 (50 g, 0.30 mol) and 50% ethyl glyoxylate (62 ml, 0.30 mol) in toluene (100 ml) was heated to 140° C. for 24 h. The progress of the reaction was monitored by TLC. After completion of the reaction, the reaction mixture was concentrated under reduced pressure and the obtained crude residue was purified by column chromatography (100-200 silica gel, 25-30% EtOAc-Hexane) to give 3 (17 g, yield: 21%) as an off-white solid (observed solid upon storing in the refrigerator over 6 h time).

¹H NMR [400 MHz, DMSO-d₆]: δ 8.35 (m, 2H), 8.2 (m, 2H), 4.52 (m, 1H), 4.1 (q, J=7.6 Hz, 2H), 3.45 (d, J=6.0 Hz, 2H), 1.7 (t, J=7.6 Hz, 3H).

LCMS: m/z: 266.17 [M−H]⁺, 92.50% (1.23 min),

Column: Kinetex EVO C18 (2.1×50 mm, 1.7ìm),

Mobile Phase: A-0.01% FA in water; B-0.01% FA in ACN,

(T/% B: 0.01/10, 0.2/10, 1.5/90, 3/90)

Flow Rate: 0.4 mL/min.

R_{f}=0.2 (Mobile phase: 50% EtOAc-Hexane).

### Step 2:

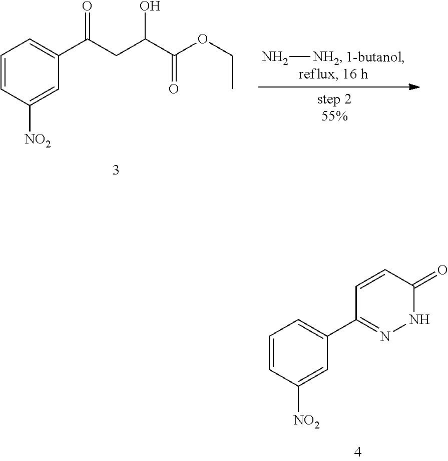

6-(3-nitrophenyl)pyridazin-3(2H)-one (4): A mixture of compound 3 (17 g, 0.063 mol), hydrazine hydrate (2.5 ml, 0.076 mol) and 1-butanol (170 ml) was stirred at 120° C. for 16 h. The progress of the reaction was monitored by TLC. After completion of the reaction, the reaction mixture was cooled to room temperature, yellow solid was formed. The yellow solid was filtered, washed with 1-butanol, and dried to afford compound 4 (7.6 g, yield: 55%) which was used in the next step without any further purification.

¹H NMR (400 MHz, DMSO-d₆): δ 9.85 (s, 1H), 8.5 (d, J=9.2 Hz, 1H), 8.4 (m, 4H), 8.12 (d, J=5.2 Hz, 1H).

LCMS: m/z: 216.15 [M−1]⁻, 90.85% (1.12 min),

Column: Kinetex EVO C18 (2.1×50 mm, 1.7ìm),

Mobile Phase: A-0.01% FA in water; B-0.01% FA in ACN,

(T/% B: 0.01/10, 0.2/10, 1.5/90, 3/90),

Flow Rate: 0.4 mL/min.

R_{f}=0.4 (Mobile phase: 30% EtOAc-Hexane).

### Step 3:

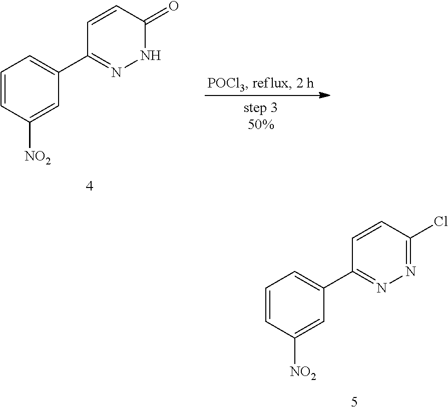 [SMILES: O=c1ccc(-c2cccc([N+](=O)[O-])c2)n[nH]1 ; O=[N+]([O-])c1cccc(-c2ccc(Cl)nn2)c1]

3-chloro-6-(3-nitrophenyl)pyridazine (5): A mixture of compound 4 (10 g, 0.046 mol) and POCl₃ (120 ml, 1.28 mol) was refluxed for 2 h. The progress of the reaction was monitored by TLC. After completion of the reaction, the reaction mixture was diluted with ice-cold water resulting in formation of a brown solid. The solid was filtered and dried under reduced pressure to afford 5 (5.5 g, yield: 50%) which was used in the next step without any further purification.

¹H NMR (400 MHz, DMSO-d₆): δ 8.5 (d, J=9.2 Hz, 1H), 8.4 (m, 4H), 8.12 (d, J=5.2 Hz, 1H).

LCMS: m/z: 236.30 [M+1]⁺, 95.02% (1.39 min),

Column: Kinetex EVO C18 (2.1×50 mm, 1.7ìm),

Mobile Phase: A-0.01% FA in water; B-0.01% FA in ACN,

(T/% B: 0.01/10, 0.2/10, 1.5/90, 3/90),

Flow Rate: 0.4 mL/min.

R_{f}=0.6 (Mobile phase: 30% EtOAc-Hexane).

### Step 4:

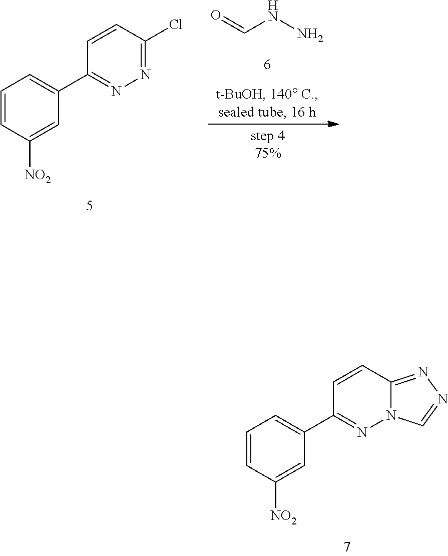

6-(3-nitrophenyl)-[1,2,4]triazolo[4,3-b]pyridazine(7): A mixture of compound 5 (5.0 g, 0.021 mol), formic-hydrazide (3.8 g, 0.063 mol) and 1-butanol (30 ml) in a sealed tube was stirred at 140° C. for 16 h. The progress of the reaction was monitored by TLC. After completion of the reaction, the reaction mixture was cooled to room temperature, and observed the formation of a yellow solid. The yellow solid was filtered and purified by silica gel column chromatography to afford compound 7 (3.85 g, yield: 75%).

¹H NMR (400 MHz, DMSO-d₆): δ 9.4 (s, 1H), 8.5 (d, J=9.2 Hz, 1H), 8.4 (m, 4H), 8.12 (d, J=5.2 Hz, 1H).

LCMS: m/z: 242.30 [M+1]⁺, 95.02% (1.10 min),

Column: Kinetex EVO C18 (2.1×50 mm, 1.7ìm),

Mobile Phase: A-0.01% FA in water; B-0.01% FA in ACN,

(T/% B: 0.01/10, 0.2/10, 1.5/90, 3/90),

Flow Rate: 0.4 mL/min.

R_{f}=0.4 (Mobile phase: 10% MeOH-DCM).

### Step 5:

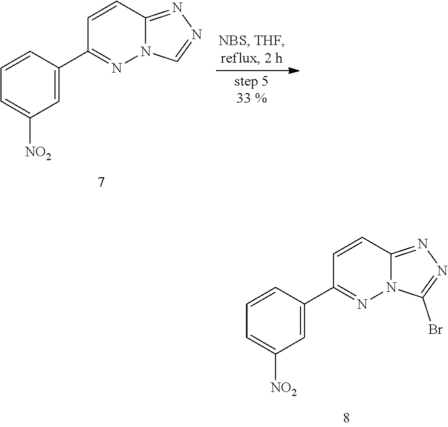 [SMILES: O=[N+]([O-])c1cccc(-c2ccc3nncn3n2)c1 ; O=[N+]([O-])c1cccc(-c2ccc3nnc(Br)n3n2)c1]

3-(3-bromo-[1,2,4]triazolo[4,3-b]pyridazin-6-yl)aniline (8): To a stirred solution of compound 7 (4.5 g, 0.018 mol) in THF (90.0 Vol) was added NBS (recrystallized from hot water) (4.0 g, 0.022 mol) in portion wise (each lot 1.0 eq up to 2.0 eq) at room temperature. The resultant reaction mixture was refluxed for 2 h. The progress of the reaction was monitored by TLC. After completion of the reaction, the reaction mixture was cooled to room temperature, quenched with NaHCO₃ solution, and extracted with ethyl acetate. The combined organic layer was dried over Na₂SO₄, filtered, and concentrated under reduced pressure to afford the crude residue which was purified by column chromatography (eluted in 1-2% MeOH-DCM) to give compound 8 (2 g, yield: 33%) as yellow color solid.

¹H NMR (400 MHz, DMSO-d₆): δ 8.9 (m, 1H), 8.6 (m, 2H), 8.47 (dd, J=1.6 Hz, 1H), 8.21 (d, J=10.0 Hz, 1H), 7.92 (m, 1H).

LCMS: m/z: 320.15 [M+1]⁺, 85.0% (1.26 min),

Column: Kinetex EVO C₁₈ (2.1×50 mm, 1.7ìm),

Mobile Phase: A-0.01% FA in water; B-0.01% FA in ACN,

(T/% B: 0.01/10, 0.2/10, 1.5/90, 3/90)

Flow Rate: 0.4 mL/min.

R_{f}=0.5 (Mobile phase: 10% MeOH-DCM).

### Step 6:

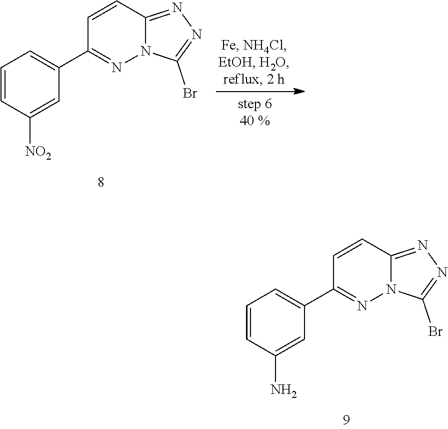 [SMILES: O=[N+]([O-])c1cccc(-c2ccc3nnc(Br)n3n2)c1 ; Nc1cccc(-c2ccc3nnc(Br)n3n2)c1]

3-(3-bromo-[1,2,4]triazolo[4,3-b]pyridazin-6-yl)aniline (9): To a stirred solution of compound 8 (1.8 g, 6.2 mmol) in ethanol:water (1:1) (50 ml) was added NH₄Cl (3 g, 62 mmol) and iron powder (3 g, 62 mmol) respectively at room temperature. The resultant reaction mixture was refluxed for 2 h. The progress of the reaction was monitored by TLC. After completion of the reaction, the hot reaction mixture was filtered through a Celite bed and washed with ethanol. The filtrate was concentrated under reduced pressure. The residue obtained was diluted with water and extracted with DCM (3×50 mL). The combined organic extracts were dried over anhydrous Na₂SO₄, filtered, and concentrated under reduced pressure. The solid obtained was purified by column chromatography (eluted in 1-2% MeOH-DCM) to afford compound 9 (700 mg, yield: 40%) as yellow color solid.

¹H NMR (400 MHz, DMSO-d₆): δ 58.9 (m, 1H), 8.6 (m, 2H), 8.47 (dd, J=1.6 Hz, 1H), 8.21 (d, J=10.0 Hz, 1H), 7.92 (m, 1H), 4.2 (s, 2H).

LCMS: m/z: 290.15 [M+1]⁺, 85.0% (1.02 min),

Column: Kinetex EVO C18 (2.1×50 mm, 1.7ìm),

Mobile Phase: A-0.01% FA in water; B-0.01% FA in ACN,

(T/% B: 0.01/10, 0.2/10, 1.5/90, 3/90)

Flow Rate: 0.4 mL/min.

R_{f}=0.4 (Mobile phase: 5% MeOH-DCM).

### Step 7:

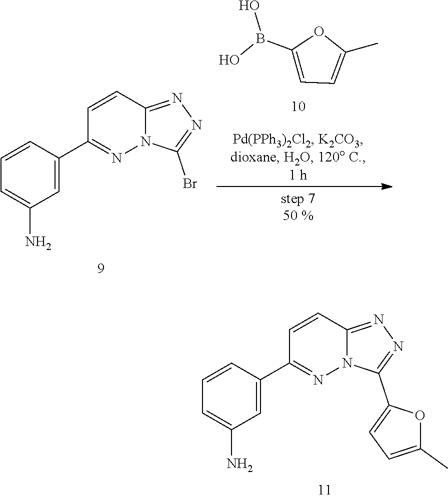

3-(3-(5-methylfuran-2-yl)-[1,2,4]triazolo[4,3-b]pyridazin-6-yl)aniline (11): To a stirred solution of compound 9 (300 mg, 1.0 mmol) in dioxane (2.0 ml) was added K₂CO₃ (500 mg, 3.0 mmol) and compound 10 (210 mg, 2.0 mol) at room temperature under argon atmosphere. The reaction mixture was degassed for 10 minutes. Pd(PPh₃)₂Cl₂ (16 mg, 0.01 mmol) was added and degassed the resultant reaction mixture for another 10 minutes. The reaction mixture was heated at 120° C. in a microwave reactor for 1 h. The progress of the reaction was monitored by TLC. After completion of the reaction, water was added to the reaction mixture and extracted with ethyl acetate. The combined organic layer was dried over Na₂SO₄, filtered and the filtrate was concentrated under reduced pressure to afford the crude. The crude residue obtained was purified by column chromatography (eluted in 2-3% MeOH-DCM) to afford compound 11 (150 mg, yield: 50%) as pale-yellow color solid.

¹H NMR [400 MHz, DMSO-d₆]: δ 8.9 (t, 1H), 8.6 (m, 2H), 8.47 (dd, J=1.6 Hz, 1H), 8.21 (d, J=10.0 Hz, 1H), 7.92 (t, 1H), 7.8 (d, J=7.2 Hz, 1H), 6.5 (d, J=2.4 Hz, 1H), 4.2 (s, 2H), 2.45 (s, 3H).

LCMS: m/z: 292.15 [M+1]⁺, 87.0% (1.02 min),

Column: Kinetex EVO C₁₈ (2.1×50 mm, 1.7ìm),

Mobile Phase: A-0.01% FA in water; B-0.01% FA in ACN,

(T/% B: 0.01/10, 0.2/10, 1.5/90, 3/90)

Flow Rate: 0.4 mL/min.

R_{f}=0.3 (Mobile phase: 10% MeOH-DCM).

### Step 8:

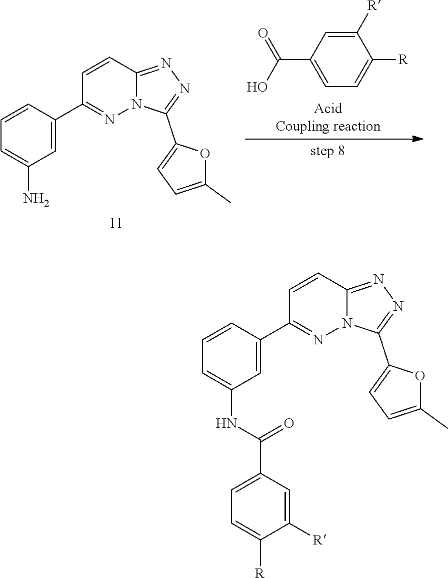

### General Procedure for Amide Couplings:

A: To a solution of acid (1.0 mol) in DM F (10.0 Vol) was added HATU (2.0 mol), DIPEA (3.0 mol), and compound 11 (1.0 mol) at room temperature and stirred for 16 hours. The reaction mixture was monitored by TLC. After completion of the reaction, the reaction mixture was diluted with ice-cold water resulting in a solid precipitate, and the obtained solid was filtered and purified by C-18 reverse phase column chromatography by eluting 0-100% ACN-water to afford the desired product.

B: To a stirred solution of acid (1.0 mol) in pyridine was added T₃P (3.0 mol), followed by compound 11 (1.0 mol) at room temperature under argon atmosphere and stirred the reaction for 16 h at 90° C. The progress of the reaction was monitored by TLC. After completion of the reaction, the reaction mixture was diluted with ice-cold water, the solid was precipitated out, and the obtained solid was filtered and purified by C-18 reverse phase column chromatography by eluting 0-100% ACN-water to afford the desired product.

3-ethyl-N-(3-(3-(5-methylfuran-2-yl)-[1,2,4]triazolo[4,3-b]pyridazin-6-yl)phenyl) benzamide was prepared according to the general procedure B:

3-ethylbenzoic acid (62 mg) & 3-(3-(5-methylfuran-2-yl)-[1,2,4]triazolo[4,3-b]pyridazin-6-yl)aniline (11) (100 mg) to afford the title compound (25 mg, Yield: 14.4%).

LCMS (m/z)=424.23 [M+H]⁺ (LCMS purity 99.20%, 3.08 min).

1H NMR (400 MHz, DMSO-d6): δ (ppm) 10.5 (s, 1H), 8.69 (s, 1H), 8.56 (d, J=9.6 Hz, 1H), 8.04 (d, J=8.0 Hz, 1H), 7.96 (d, J=10.0 Hz, 1H), 7.92 (d, J=8.0 Hz, 1H), 7.8 (m, 2H), 7.6 (m, 2H), 7.5 (m, 2H), 6.5 (d, J=3.2 Hz, 1H), 2.71 (q, J=7.6 Hz, 2H), 2.47 (s, 3H), 1.26 (t, J=7.6 Hz, 3H).

3-ethyl-4-fluoro-N-(3-(3-(5-methylfuran-2-yl)-[1,2,4]triazolo[4,3-b]pyridazin-6-yl)phenyl)benzamide was prepared according to the general procedure A:

3-ethyl-4-fluorobenzoic acid (69 mg) & 3-(3-(5-methylfuran-2-yl)-[1,2,4]triazolo[4,3-b]pyridazin-6-yl)aniline (11) (120 mg) to afford the title compound (30 mg, Yield: 16.60%).

LCMS (m/z)=442.13 [M+H]⁺ (LCMS purity 98.83%, 3.12 min)

1H NMR (400 MHz, DMSO-d6) δ (ppm) 10.5 (s, 1H), 8.66 (s, 1H), 8.54 (d, J=10.0 Hz, 1H), 8.0-7.9 (m, 5H), 7.6 (m, 2H), 7.3 (m, 1H), 6.5 (d, J=3.2 Hz, 1H), 2.71 (q, J=7.6 Hz, 2H), 2.47 (s, 3H), 1.25 (t, J=7.6 Hz, 3H).

3-chloro-N-(3-(3-(5-methylfuran-2-yl)-[1,2,4]triazolo[4,3-b]pyridazin-6-yl) phenyl)benzamide was prepared according to the general procedure A:

3-chlorobenzoic acid (81 mg) & 3-(3-(5-methylfuran-2-yl)-[1,2,4]triazolo[4,3-b]pyridazin-6-yl)aniline (11) (150 mg) to afford the title compound (30 mg, Yield: 13.6%).

LCMS (m/z)=430.11 [M+H]⁺ (LCMS purity 95.64%, 6.32 min)

¹H NMR (400 MHz, DMSO-d6): δ (ppm): 10.61 (s, 1H), 8.67 (s, 1H), 8.55 (d, J=9.6 Hz, 1H), 7.96 (m, 5H), 7.62 (m, 4H), 6.5 (s, 1H), 2.45 (s, 3H).

3-chloro-4-fluoro-N-(3-(3-(5-methylfuran-2-yl)-[1,2,4]triazolo[4,3-b]pyridazin-6-yl)phenyl)benzamide was prepared according to the general procedure A:

3-chloro-4-fluorobenzoic acid (90 mg) & 3-(3-(5-methylfuran-2-yl)-[1,2,4]triazolo[4,3-b]pyridazin-6-yl)aniline (11) (150 mg) to afford the title compound (30 mg, Yield: 13.0%).

LCMS (m/z)=448.17 [M+H]⁺ (LCMS purity 99.1%, 3.08 min)

¹H NMR (400 MHz, DMSO-d6): δ (ppm): 10.63 (s, 1H), 8.66 (s, 1H), 8.53 (d, J=10.4 Hz, 1H), 8.24 (m, 1H), 8.03 (m, 4H), 7.60 (m, 3H), 6.51 (d, J=2.4 Hz, 1H), 2.47 (s, 3H).

4-fluoro-N-(3-(3-(5-methylfuran-2-yl)-[1,2,4]triazolo[4,3-b]pyridazin-6-yl)phenyl) benzamide was prepared according to the general procedure A:

4-fluorobenzoic acid (72 mg) & 3-(3-(5-methylfuran-2-yl)-[1,2,4]triazolo[4,3-b]pyridazin-6-yl)aniline (11) (150 mg) to afford the title compound (60 mg, Yield: 28.16%).

LCMS (m/z)=414.46 [M+H]⁺ (LCMS purity 95.73%, 2.82 min)

¹H NMR (400 MHz, DMSO-d6): δ (ppm): 10.57 (s, 1H), 8.67 (s, 1H), 8.55 (d, 1H, J=10.0 Hz), 8.11 (m, 2H), 8.0 (m, 3H), 7.61 (m, 2H), 7.41 (m, 2H), 6.50 (d, 1H, J=2.8 Hz), 2.47 (s, 3H).

3-(isopropylthio)-N-(3-(3-(5-methylfuran-2-yl)-[1,2,4]triazolo[4,3-b]pyridazin-6-yl)phenyl)benzamide was prepared according to the general procedure A:

3-(isopropylthio)benzoic acid (67 mg) & 3-(3-(5-methylfuran-2-yl)-[1,2,4]triazolo[4,3-b]pyridazin-6-yl)aniline (11) (100 mg) to afford the title compound (25 mg, Yield: 15.5%).

LCMS (m/z)=470.18 [M+H]⁺ (LCMS purity 98.69%, 3.24 min)

¹H NMR (400 MHz, DMSO-d6) δ (ppm): 10.57 (s, 1H), 8.67 (s, 1H), 8.56 (d, J=10.0 Hz, 1H), 8.0-7.86 (m, 4H), 7.8 (d, J=7.2 Hz, 1H), 7.6 (m, 4H), 6.5 (d, J=2.4 Hz, 1H), 3.6 (m, 1H), 2.5 (s, 3H), 1.29 (d, J=6.0 Hz, 6H).

### Synthesis of 4-fluoro-N-(3-(3-(thiazol-5-yl)-[1,2,4]triazolo[4,3-b]pyridazin-6-yl)phenyl)benzamide

3-(6-chloropyridazin-3-yl) aniline (12): To a stirred solution of compound 5 (0.5 g, 2.21 mmol) in ethanol:water (1:1) (12 ml) was added NH₄Cl (1.14 g, 21.22 mmol) and iron powder (1.19 g, 21.22 mmol) at room temperature. The resultant reaction mixture was refluxed for 2 h. The progress of the reaction was monitored by TLC. After completion of the reaction, the hot reaction mixture was filtered through a Celite bed and washed with ethanol. The filtrate was concentrated under reduced pressure. The residue obtained was diluted with water and extracted with DCM (3×30 mL). The combined organic extracts were dried over anhydrous Na₂SO₄, filtered and the filtrate was concentrated under reduced pressure. The solid was purified by column chromatography (eluted in 1-2% MeOH-DCM) to afford compound 12 (185 mg, yield: 42%) as yellow color solid.

¹H NMR (400 MHz, DMSO-d₆): δ 8.34 (s, 1H), 8.23 (d, J=8.8 Hz, 1H), 8.0 (d, J=9.2 Hz, 1H), 7.69 (d, J=8.0 Hz, 1H), 7.6 (d, J=8.0 Hz, 1H), 7.44 (t, 1H), 4.2 (s, 2H).

LCMS: m/z: 206.15 [M+1]⁺, 95.64% (1.06 min),

Column: Kinetex EVO C18 (2.1×50 mm, 1.7ìm),

Mobile Phase: A-0.01% FA in water; B-0.01% FA in ACN,

(T/% B: 0.01/10, 0.2/10, 1.5/90, 3/90)

Flow Rate: 0.4 mL/min.

3-(3-(thiazol-5yl)-[1,2,4]triazolo[4,3-b]pyridazin-6-yl)aniline (14): A mixture of compound 12 (0.870 mmol), hydrazine derivative 13 (2.63 mmol) and 1-butanol (10.0 Vol) in a sealed tube was stirred at 140° C. for 16 h. The progress of the reaction was monitored by TLC. After completion of the reaction, the reaction mixture was cooled to room temperature and provided a precipitate. The precipitate was filtered, washed with 1-butanol, and used in the next step without further purification.

LCMS: m/z: 295.15 [M+1]⁺, 43.57% (1.06 min),

Column: Kinetex EVO C18 (2.1×50 mm, 1.7ìm),

Mobile Phase: A-0.01% FA in water; B-0.01% FA in ACN,

(T/% B: 0.01/10, 0.2/10, 1.5/90, 3/90)

Flow Rate: 0.4 mL/min.

Step-11: 4-fluoro-N-(3-(3-(thiazol-5-yl)-[1,2,4]triazolo[4,3-b]pyridazin-6-yl)phenyl)benzamide was prepared according to the general procedure (A):

Acid (48 mg) & Compound 14 (100 mg) to afford the title compound (11.12 mg, Yield: 08%).

LCMS (m/z)=417.16 [M+H]⁺ (LCMS purity 98.76%, 2.51 min)

¹H NMR (400 MHz, DMSO-d6) δ (ppm): 10.56 (s, 1H), 9.35 (s, 1H), 9.01 (s, 1H), 8.63 (s, 1H), 8.56 (d, J=10 Hz, 1H), 8.0 (m, 5H), 7.59 (t, J=8.0 Hz, 1H), 7.37 (t, J=8.8 Hz, 2H).

### Preparation of Compound 102 By the Method of Scheme 2

### Step 1:

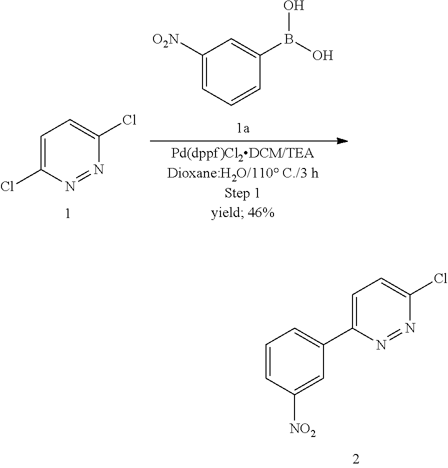 [SMILES: O=[N+]([O-])c1cccc(B(O)O)c1 ; Clc1ccc(Cl)nn1 ; O=[N+]([O-])c1cccc(-c2ccc(Cl)nn2)c1]

3-chloro-6-(3-nitrophenyl)pyridazine (2): To a stirred solution of 3,6-dichloropyridazine-1 (107.2 g, 595.88 mmol) in dioxane (600 mL), water (150.00 mL) was added and Et₃N (259.16 mL, 1796 mmol) followed by the addition of (3-nitrophenyl)boronic acid-1a (100 g, 664 mmol) at RT under argon atmosphere. The reaction mixture was degassed for 20 minutes. After 20 min, Pd(dppf)Cl2·DCM (6.84 g, 8.36 mmol) was added to the reaction mixture and degassed the reaction mass for another 10 minutes. After 10 min, the reaction mixture was heated at 110° C. in an round bottom flask for 3 h. TLC indicated completion of the starting material. Then, the reaction mass was cooled to RT, the resulting solid precipitate was filtered, washed twice with water (2×200 mL) and dried to afford the crude product. Then the crude product was purified by column chromatography using 100-200 mesh size silica gal (eluted in 100% DCM) to afford 3-chloro-6-(3-nitrophenyl)pyridazine (2, 68 g, yield: 46%) as Off white solid. LCMS: m/z 236.0 (M+H). ¹H NMR (400 MHz, DMSO-d₆): δ 8.96 (t, J=1.6 Hz, 1H), 8.61 (d, J=8.0 Hz, 1H), 8.56 (d, J=9.2 Hz, 1H), 8.42 (dd, J=2.4, 8.4 Hz, 1H), 7.88 (t, J=8.4 Hz, 2H).

HPLC: Rt=6.80 min, 95.00%

### Step 2:

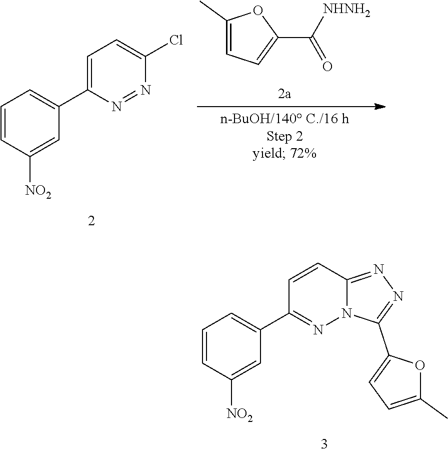

### 3-(5-methylfuran-2-yl)-6-(3-nitrophenyl)-[1,2,4]triazolo[4,3-b]pyridazine(3)

A solution of 3-chloro-6-(3-nitrophenyl) pyridazine-2 (68 g, 287.58 mmol) and 5-methylfuran-2-carbohydrazide (2a, 44.48 g, 317.31 mmol) in n-butanol (600 mL) was stirred at 140° C. for 16 h. TLC indicated completion of the starting material. Then, the reaction mass was cooled to RT, and the resulting precipitate was filtered. The filtered residue was washed with a combination of EtOAc:DCM (7:3) to afford 3-(5-methylfuran-2-yl)-6-(3-nitrophenyl)-[1,2,4]triazolo[4,3-b]pyridazine (68 g, yield: 72%) as yellow solid. LCMS: m/z 322.1 (M+H). ¹H NMR (400 MHz, DMSO-d₆): δ 8.94 (t, J=2.0 Hz, 1H), 8.67 (d, J=9.6 Hz, 1H), 8.62 (d, J=10.0 Hz, 1H), 8.47 (dd, J=1.2, 8.0 Hz, 1H), 8.19 (d, J=9.6 Hz, 1H), 7.95 (t, J=8.0 Hz, 1H), 7.52 (d, J=3.2 Hz, 1H), 6.51 (d, J=2.4 Hz, 1H), 2.47 (s, 3H). HPLC: Rt=6.79 min, 98.04%

### Step 3:

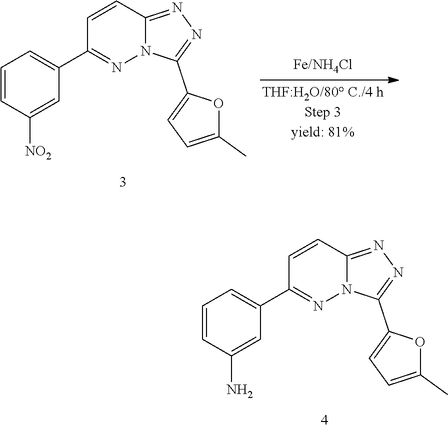 [SMILES: Cc1ccc(-c2nnc3ccc(-c4cccc([N+](=O)[O-])c4)nn23)o1 ; "THF:H2O/80° C./4 hStep 3yield: 81%" ; Cc1ccc(-c2nnc3ccc(-c4cccc(N)c4)nn23)o1]

### 3-(3-(5-methylfuran-2-yl)-[1,2,4]triazolo[4,3-b]pyridazin-6-yl)aniline(4)

To a stirred solution of 3-(5-methylfuran-2-yl)-6-(3-nitrophenyl)-[1,2,4]triazolo[4,3-b]pyridazine-3 (68 g, 211.65 mmol) in TH F (350 mL) and water (350 mL) was added iron powder (59.08 g, 1058 mmol) and ammonium chloride (56.61 g, 1058 mmol) at RT. The reaction mixture was heated at 80° C. for 4 h. TLC indicated disappearance of the starting material. Then, the reaction mass was cooled to RT and filtered over sintered funnel. The filtrate was diluted with water and extracted with DC M (3×500 ml). The combined organic layers were dried over sodium sulfate and concentrated under reduced pressure. Residue obtained was washed with the combination of DCM:EtOAc:MeOH (7:2:1) to afford 3-(3-(5-methylfuran-2-yl)-[1,2,4]triazolo[4,3-b]pyridazin-6-yl)aniline (51.38 g, yield: 81%) as yellow solid. LCMS: m/z 292.1 (M+H). ¹H NMR (400 MHz, DMSO-d₆): δ 8.47 (d, J=9.6 Hz, 1H), 7.88 (d, J=10.0 Hz, 1H), 7.54 (d, J=3.2 Hz, 1H), 7.39 (s, 1H), 7.28-7.22 (m, 2H), 6.80 (d, J=7.2 Hz, 1H), 6.48 (d, J=2.0 Hz, 1H), 5.46 (s, 2H), 2.47 (s, 3H). HPLC: Rt=6.21 min, 97.25%

### Step 4:

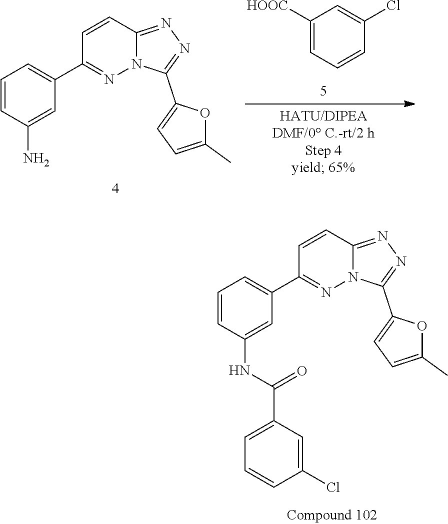 [SMILES: Cc1ccc(-c2nnc3ccc(-c4cccc(N)c4)nn23)o1 ; O=C(O)c1cccc(Cl)c1 ; Cc1ccc(-c2nnc3ccc(-c4cccc(NC(=O)c5cccc(Cl)c5)c4)nn23)o1]

### 3-chloro-N-(3-(3-(5-methylfuran-2-yl)-[1,2,4]triazolo[4,3-b]pyridazin-6-yl)phenyl)benzamide (Compound 102)

To a stirred solution of 3-chloro-benzoic acid (30.36 g, 194.45 mmol) in DM F (500 mL) was added DIPEA (92.46 ml, 528.34 mmol) and HATU (87.14 g, 229.32 mmol) at 0° C. The reaction mixture was stirred at the same temperature for 30 minutes, then 3-(3-(5-methylfuran-2-yl)-[1,2,4]triazolo[4,3-b]pyridazin-6-yl)aniline (51.38 g, 176.16 mmol) was added at same temperature. The reaction mixture was stirred at RT under argon atmosphere for 3 h. After 3 h TLC indicated disappearance of the starting material. Then, the reaction mass was cooled to RT, diluted with ice-cold water (500 mL), the resulting solid precipitate was filtered and dried to provide crude product. The crude solid was taken in 50% EtOAc in DCM (500 mL) and stirred for 15 minutes then filtered. The resulting pale brown solids were added to 30% MeOH in CH₃CN and was heated at 50° C. for 1 h in water-bath. The solid was filtered and dried to get 3-chloro-N-(3-(3-(5-methylfuran-2-yl)-[1,2,4]triazolo[4,3-b]pyridazin-6-yl)phenyl)benzamide (51.46 g, yield: 65%). LCMS: m/z 430.1 (M+H). ¹H NMR (400 MHz, DMSO-d₆): δ 10.63 (s, 1H), 8.68 (s, 1H), 8.56 (d, J=9.6 Hz, 1H), 8.06-7.96 (m, 5H), 7.73-7.59 (m, 4H), 6.51 (s, 1H), 2.47 (s, 3H). HPLC: Rt=7.33 min, 95.74%.

### Step 2a:

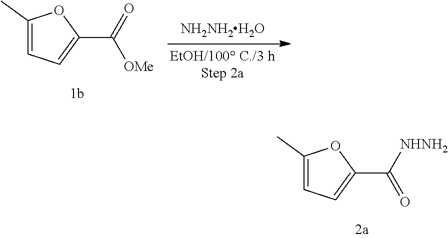

### Synthesis of 5-methylfuran-2-carbohydrazide (2a)

To a stirred solution of methyl 5-methylfuran-2-carboxylate (50 g, 357 mmol) in ethanol (500 mL) was added hydrazine monohydrate (87 mL, 1784 mmol). The reaction mixture was brought to reflux at 100° C. for 3 h. After 3 h TLC indicated absence of the starting material. The reaction mass was cooled to RT and evaporated under reduced pressure. Azeotropic distillation was conducted with toluene (5 times 50 mL) and dried under high vacuum to afford 5-methylfuran-2-carbohydrazide (51.00 g, yield: 98%) as orange color solid. LCMS: m/z 141.1 (M+H). ¹H NMR (400 MHz, DMSO-d₆): δ 9.46 (s, 1H), 6.96 (d, J=3.2 Hz, 1H), 6.21 (d, J=3.2 Hz, 1H), 4.35 (s, 2H), 2.30 (s, 3H). HPLC: Rt=2.03 min, 95.68%.

### Preparation of Compound 119 According to Schemes 2 and 3

### Step 1:

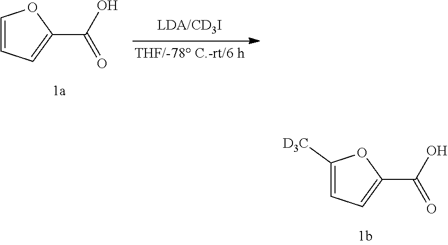 [SMILES: O=C(O)c1ccco1 ; [2H]C([2H])([2H])c1ccc(C(=O)O)o1]

### 5-(methyl-d3) furan-2-carboxylic acid (1b)

To a stirred solution of furan-2-carboxylic acid (10.00 g, 89.221 mmol) in TH F (100.0 ml) was added LDA (111 ml, 223.05 mmol) at −78° C. After stirring at 0° C. for 30 min, reaction mixture was cooled at −78° C. and added CD₃I (25.86 g, 178.44 mmol). The reaction mixture was brought to room temperature and stirred for 6 h. Then, the reaction mixture was quenched with ammonium chloride solution, acidified with HCl and extracted with EtOAc. The EtOAc extract was dried over Na₂SO₄ and concentrated under vacuum to get crude product. Then the crude material was purified by SFC (Column; (Chiralpak IC, 250 mm×21 mm, 5 μm), Mobile Phase A: CO₂, Phase B: 0.1% HCOOH in EtOH, Gradient Elusion), to afford 5-(methyl-d3) furan-2-carboxylic acid (3.60 g, yield: 31%) as light-yellow solid. ¹H NMR: (400 MHz, DMSO-d6): δ 12.80 (s, 1H), 7.1 (q, J=3.37 Hz, 1H), 6.29 (d, J=3.37 Hz, 1H). HPLC: RT=5.156 min, 97.602%.

### Step 2:

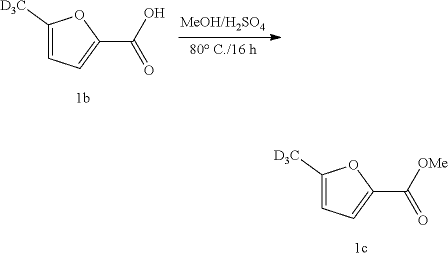 [SMILES: [2H]C([2H])([2H])c1ccc(C(=O)O)o1 ; [2H]C([2H])([2H])c1ccc(C(=O)OC)o1]

Methyl 5-(methyl-d3) furan-2-carboxylate (1c):

To the stirred solution of 5-(methyl-d3) furan-2-carboxylic acid (3.5 g, 27.1 mmol) in methanol (35 ml) was added H₂SO₄ (1.4 ml) and the reaction mixture was heated under reflux at 80° C. for 16 hours. TLC indicated absence of the starting material. The reaction mixture was cooled to RT and evaporated under reduced pressure. The crude product was basified with NaHCO₃ solution and extracted with EtOAc. The EtOAc extract dried over Na₂SO₄ and evaporated under reduced pressure. Then the crude product was purified by column chromatography using silica gel (60-120 mesh) (eluted in 20-25% EtOAc-Hexane) to afford methyl 5-(methyl-d3) furan-2-carboxylate (3.5 g, yield: 90%) as light brown liquid. LCMS: m/z 144.15 (M+H). ¹H NMR: (400 MHz, DMSO d6): δ 7.2 (d, J=3.37 Hz, 1H), 6.33 (d, J=3.37 Hz, 1H), 3.78 (s, 3H).

### Step 3:

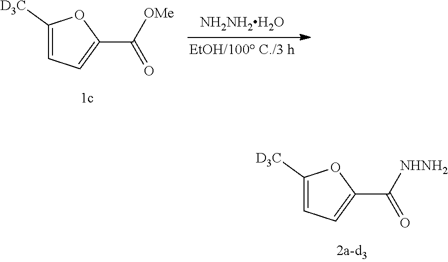

### 5-(methyl-d3) furan-2-carbohydrazide (2a-d3)

To a stirred solution of methyl 5-(methyl-d3) furan-2-carboxylate (3.5 g, 24.4481 mmol) in ethanol (35 mL) was added hydrazine monohydrate (4.895 g, 97.7926 mmol). The reaction mixture was heated under reflux at 100° C. for 3 h. After 3 h TLC indicated absence of the starting material. The reaction mixture was cooled to RT, evaporated under reduced pressure and the crude material was purified by column chromatography using silica gel (60-120 mesh) (eluted in 4-6% MeOH-DCM) to afford 5-(methyl-d3) furan-2-carbohydrazide (3.5 g, yield: 99%) as white solid. LCMS: m/z 144.15 (M+H). ¹H NMR (400 MHz, DMSO-d₆): δ 9.45 (m, 1H), 6.96 (d, J=3.37 Hz, 1H), 6.2 (d, J=3.23 Hz, 1H), 4.35 (s, 2H). HPLC: RT=5.207 min, 95.07%.

### Step 4:

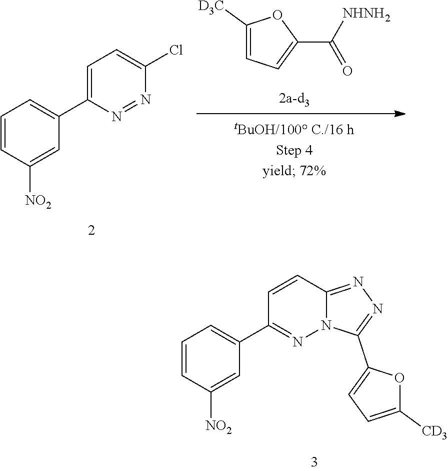 [SMILES: O=[N+]([O-])c1cccc(-c2ccc(Cl)nn2)c1 ; [2H]C([2H])([2H])c1ccc(C(=O)NN)o1 ; [2H]C([2H])([2H])c1ccc(-c2nnc3ccc(-c4cccc([N+](=O)[O-])c4)nn23)o1]

### 3-(5-(methyl-d3) furan-2-yl)-6-(3-nitrophenyl)-[1,2,4]triazolo[4,3-b]pyridazine (3)

To the stirred solution of 3-chloro-6-(3-nitrophenyl) pyridazine (2, 0.5 g, 2.121 mmol) in Butanol (10 ml) was added 5-(methyl-d3) furan-2-carbohydrazide (0.334 g, 2.334 mmol) at room temperature and the solution was heated at 140° C. for 16 h. TLC indicated absence of the starting material. The reaction mixture was cooled to RT, the resulting solid precipitate was filtered, and the residue obtained was washed with a combination of DCM:EtOAc:MeOH (7:2:1) to afford 3-(5-(methyl-d3) furan-2-yl)-6-(3-nitrophenyl)-[1,2,4]triazolo[4,3-b]pyridazine (0.470 g, yield: 68%) as yellow solid. LCMS: m/z 325.05 (M+H). ¹H NMR (400 M H, DMSO-cd): δ 8.94 (t, J=1.91 Hz, 1H), 8.62 (m, 2H), 8.46 (m, 1H), 8.17 (d, J=9.83 Hz, 1H), 7.98 (t, J=8 Hz, 1H), 7.51 (d, J=3.37 Hz, 1H), 6.51 (d, J=3.37 Hz, 1H). HPLC: RT=7.323 min, 96.64%.

### Step 5:

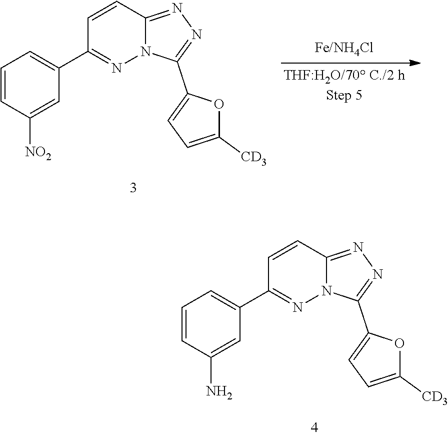 [SMILES: [2H]C([2H])([2H])c1ccc(-c2nnc3ccc(-c4cccc([N+](=O)[O-])c4)nn23)o1 ; [2H]C([2H])([2H])c1ccc(-c2nnc3ccc(-c4cccc(N)c4)nn23)o1]

### 3-(3-(5-(methyl-d3) furan-2-yl)-[1,2,4]triazolo[4,3-b]pyridazin-6-yl) aniline (4)

To a stirred solution of 3-(5-(methyl-d3) furan-2-yl)-6-(3-nitrophenyl)-[1,2,4]triazolo[4,3-b]pyridazine (0.470 g, 1.449 mmol) in THF:water (3:1) (20 ml) was added iron powder (0.404 g, 7.2461 mmol), ammonium chloride (0.387 g, 7.2461 mmol) and the reaction mixture was heated at 80° C. for 2 h. TLC indicated absence of the starting material. The reaction mixture was cooled to RT and filtered over a sintered funnel. The filtrate was diluted with water and extracted with DCM (3 times 25 ml). The combined organic layers were dried over sodium sulphate and concentrated under reduced pressure. Residue obtained was washed with the combination of DCM:EtOAc:MeOH (7:2:1) to afford 3-(3-(5-(methyl-d3) furan-2-yl)-[1,2,4]triazolo[4,3-b]pyridazin-6-yl) aniline (0.390 g, yield: 91%) as yellow solid. LCMS: m/z 295.0 (M+H). ¹H NMR (400 MHz, DMSO-d): δ 8.47 (d, J=9.68 Hz, 1H), 7.88 (d, J=9.68 Hz, 1H), 7.54 (d, J=3.23 Hz, 1H), 7.46 (s, 1H), 7.34 (br d, J=7.48 Hz, 1H), 7.28 (m, 1H), 6.85 (br d, J=7.78 Hz, 1H), 6.48 (d, J=3.23 Hz, 1H). HPLC: RT=5.901 min, 95.275%.

### Step 6:

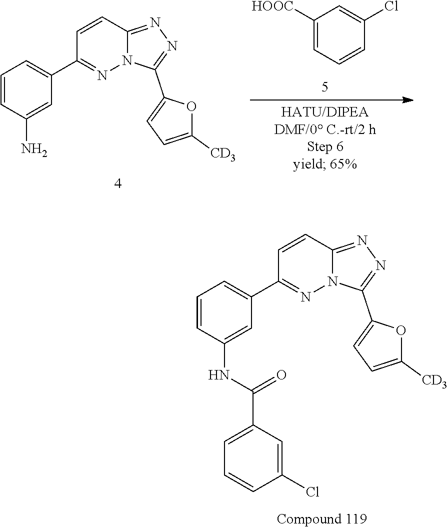 [SMILES: [2H]C([2H])([2H])c1ccc(-c2nnc3ccc(-c4cccc(N)c4)nn23)o1 ; O=C(O)c1cccc(Cl)c1 ; [2H]C([2H])([2H])c1ccc(-c2nnc3ccc(-c4cccc(NC(=O)c5cccc(Cl)c5)c4)nn23)o1]

### 3-chloro-N-(3-(3-(5-(methyl-d3) furan-2-yl)-[1,2,4]triazolo[4,3-b]pyridazin-6-yl) phenyl) benzamide (Compound 119)

To a stirred solution of 3-chloro-4-fluorobenzoic acid (0.063 g, 0.4077 mmol) in DM F (2 mL) was added HATU (0.258 g, 0.6795 mmol) at 0° C. The reaction mixture was stirred at the same temperature for 30 minutes, then 3-(3-(5-(methyl-d3) furan-2-yl)-[1,2,4]triazolo[4,3-b]pyridazin-6-yl) aniline (0.100 g, 0.3397 mmol) and DIPEA (0.17 mL, 1.0192 mmol) were added. The reaction mixture was stirred at RT under argon atmosphere for 3 h. After 3 h TLC indicated disappearance of the starting material Then, the reaction mixture was cooled to RT, diluted with ice-cold water (100 mL), solid precipitate was filtered and dried to provide crude product. The crude solid was taken in 20 ml of DCM:EtOAc:MeOH (7:2:1) mixture and was heated at 50° C. for 1 h in water-bath. The solid was filtered, washed with MeOH and dried to provide 3-chloro-N-(3-(3-(5-(methyl-d3) furan-2-yl)-[1,2,4]triazolo[4,3-b]pyridazin-6-yl) phenyl) benzamide (0.030 g, yield: 20%) as yellow solid. LCMS: m/z 433.1 (M+H). ¹H NMR (400 MHz, DMSO-d₆): δ 10.63 (s, 1H), 8.68 (s, 1H), 8.55 (d, J=9.68 Hz, 1H), 8.0 (m, 5H), 7.71 (d, J=8.23 Hz, 1H), 7.61 (m, 3H), 6.51 (d, J=3.23 Hz, 1H). HPLC: RT=6.935 min, 97.573%.

### Pharmacology

### In Vitro Assays

### 1. Cell Viability Assay (MCF7)

In order to determine a compound's effect on cell viability, PrestoBlue assays were performed as previously described by Kuhn et al. (2013) with modifications. MCF-7 human breast cancer and IGR-1 human melanoma cell lines were seeded into 384-well plates 24 hours prior to addition of drug. Cells are treated with 0 to 100 μM (concentrations) of compound solubilized in DMSO, adjusting the final concentration of DMSO to 0.5% in the well. Three days after drug treatment, cell viability was measured by adding Cell Titer Glo (Promega.; cat. No. G7570) according to the manufacturer's instructions. Briefly, Cell Titer Glo Substrate is diluted in Cell Titer Glo Buffer making the Cell Titer Glo Reagent. Cell Titer Glo Reagent is added to each well, mixed for 2 minutes to lyse cell, and then luficerase signal is read on a plate reader. Data is analyzed using the GraphPad Prism software (GraphPad Software, Inc.), and IC50 (dose leading to 50% cell death) was calculated from the dose-response curves. The percentage of living cells was then computed by comparison with control wells. Kuhn, Jonas et al., Assay and Drug Development Technologies, March 2013, Label-Free Cytotoxicity Screening Assay by Digital Holographic Microscopy.

### 2. Rac Activation AlphaScreen Assay (RacI AS)

AlphaScreen® assays were performed in 96-well microplates in a final reaction volume of 60 μL. Recombinant His-Rac1, recombinant GST-PBD (PAK Binding Domain), donor and acceptor beads (PerkinElmer), and inhibitors were incubated in exchange buffer (20 mM Tris pH 7.5, 50 mM NaCl, 1 mM MgCl₂, 1 mM EDTA, 500 nM GTPyS (guanosine 5′-[Y-thio]triphosphate)) at 37° C. Readings were performed on a Tecan M 1000 pro microplate reader after 1 hour. Data was analyzed using the GraphPad Prism software (GraphPad Software, Inc.), and IC₅₀ (dose leading to 50% disruption of complex) was calculated from the dose-response curves.

### 3. Cell Migration Assay

An essential characteristic of malignant cells is their ability to migrate, invade host tissues and to produce metastases. In order to evaluate the capacity of one compound to affect the ability of tumoral cells to migrate, migration assays are performed using HUV EC cells.

MCF7 (2.5×10 4) are seeded onto uncoated filters in a 24-well transwell Boyden chamber (8-mm pore size; Costar) and allowed to migrate in the presence and absence of different doses of the Rac1 inhibitor test material (5, 6.25, 10, 12.5, 20, 25 μM). The cells that migrated to the underside of the filter are stained with crystal violet and counted under the bright field microscopy.

### 4. Western Blot Analysis

Western blot analysis is used to identify specific proteins from a complex mixture of proteins extracted from cells. Equal amount of protein is run on the SDS-PAG E gel and after separating the protein mixture, it is transferred to a membrane. The transferred protein is then probed with a combination of antibodies: one antibody specific to the protein of interest (primary antibody) and another antibody specific to the host species of the primary antibody (secondary antibody). The secondary antibody is complexed with an enzyme, which when combined with an appropriate substrate, will produce a detectable signal.

MCF7 cells are treated with increasing concentrations of test material for two hours. SDS-PAG E are conducted on cell lysates and Western Blot analysis are conducted on samples for total and phosphorylated AKT, MEK 1/2, and ERK 1/2.

### 5. Mouse Liver Microsome (MLM) Stability Assay

Incubation of test compounds with mouse liver microsomes in the presence of NADPH co-factor was used to assay the in vitro mouse liver microsome metabolic clearance rate. In the presence of the co-factor NADPH, which initiates the reaction, mouse liver microsomes were incubated with test compounds at 37° C. at fixed time intervals. After centrifugation, supernatants were measured by LC/M S. The amount of compound at each time interval was compared to the amount of compound present at the 0 minute timepoint and percentage compound remaining values were calculated. MLM intrinsic clearance (Clint) (μL/min/mg protein) is calculated based on compound half-life.

Compounds in Table 6 having a 5-methyl-furanyl group or thiazolyl group at the R¹ position have superior Rac1 inhibitory activity and cell growth inhibitory activity against the MCF7 cell line compared with the 5-methyl-thienyl compounds and other related compounds. Compounds in Table 6 having a 5-methyl-furanyl group or thiazolyl group at the R¹ position also have superior MLM activity compared with other structurally similar Rac1 inhibitors. Other examples of compounds where the MLM clearance is below 50 μL/min/mg protein include the following: 104, 106, 110, 111, 113, 114, 115 and 117.

TABLE 6
Biological Activity of Select Compounds

| | Rac1 IC₅₀ | MCF7 IC₅₀ | MLM Clearance |
|---|---|---|---|
| Compound No. | (μM) | (μM) | (μL/min/mg protein) |
| (102) | 0.52 | 1.4 | 19.7 |
| (103) | 0.43 | 2.3 | 4.5 |
| (100) | 0.08 | 11.3 | NA |
| (106) | 0.27 | 3.5 | 28.2 |
| (105) | 0.15 | 2.0 | 38.7 |
| (119) | 1.5 | 6.3 | |
| (201) | 0.17 | 3.1 | 15.5 |
| (200) | 0.24 | 1.8 | 54 |
| (303) | >5 | >10 | N/A |
| (304) | 0.69 | 19.8 | N/A |
| Comparator 1 | >5 | | N/A |
| Comparator 2 | >5 | | N/A |
| Comparator 3 | >5 | | N/A |

The disclosed compounds in general have no or minimal activity against c-M et. Representative compounds were tested in a commercial kinase screen of over 90 kinases (KinaseScan by Eurofins Discovery). Representative Compound 102 was inactive against all kinases tested, including c-M et, at a concentration that was substantially inhibitory for Rac1.

1. A compound of Formula I: 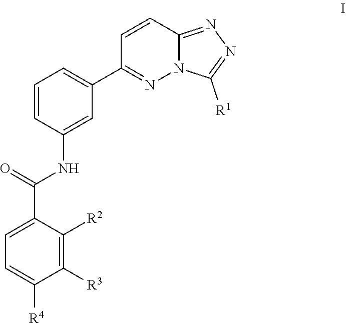
or a pharmaceutically acceptable salt thereof, wherein:
R¹ is a 5-membered heteroaryl selected from furanyl, oxazolyl, isoxazolyl, pyrrolyl, pyrazolyl or thiazolyl wherein R¹ is optionally substituted by C₁₋₃ alkyl, C₁₋₃ hydroxyalkyl, C₁₋₃alkylcarbonyl, C₁₋₃ haloalkyl or C₁₋₃ haloalkylcarbonyl;
R² is H or F;
R³ is hydrogen, halo, C₁₋₅ alkyl, —S(C₁₋₄ alkyl), C₁₋₅ haloalkyl, C₁₋₄ alkoxy, —CO(C₁₋₄ alkyl), —CONH(C₁₋₄ alkyl), —CON(C₁₋₄ alkyl)₂, —CO₂(C₁₋₄ alkyl), —NH(C₁₋₄ alkyl), —N(C₁₋₄ alkyl)₂, or C₁₋₄ haloalkoxy; and
R⁴ is H or F.

2. The compound of claim 1, or a pharmaceutically acceptable salt thereof, wherein R¹ is furanyl optionally substituted by C₁₋₃ alkyl, C₁₋₃ hydroxyalkyl, C₁₋₃alkylcarbonyl, C₁₋₃ haloalkyl or C₁₋₃ haloalkylcarbonyl.

3. The compound of claim 1, or a pharmaceutically acceptable salt thereof, wherein R¹ is 5-methyl-furan-2-yl.

4. The compound of claim 3, or a pharmaceutically acceptable salt thereof, wherein R² is H and R³ is hydrogen, halo, C₁₋₅ alkyl, or C₁₋₄ alkoxy.

5. The compound of claim 3, or a pharmaceutically acceptable salt thereof, wherein R² is H and R³ is hydrogen, chloro or fluoro.

6. The compound of claim 1, or a pharmaceutically acceptable salt thereof, wherein R¹ is thiazole optionally substituted by C₁₋₃ alkyl, C₁₋₃ hydroxyalkyl, C₁₋₃alkylcarbonyl, C₁₋₃ haloalkyl or C₁₋₃ haloalkylcarbonyl.

7. The compound of claim 1, or a pharmaceutically acceptable salt thereof, wherein R¹ is thiazole.

8. The compound of claim 6, or a pharmaceutically acceptable salt thereof, wherein R² is H and R³ is hydrogen, halo, C₁₋₅ alkyl, or C₁₋₄ alkoxy.

9. The compound of claim 6, or a pharmaceutically acceptable salt thereof, wherein R² is H and R³ is hydrogen, methoxy, chloro or fluoro.

10. The compound of claim 6, or a pharmaceutically acceptable salt thereof, wherein R² is H and R³ is hydrogen or methoxy.

11. A pharmaceutical composition comprising: i) a pharmaceutically acceptable carrier, diluent or excipient; and ii) and the compound of claim 1 or a pharmaceutically acceptable salt thereof.

12. A method of treating cancer in a patient comprising administering to the patient an effective amount of the compound of claim 1 or a pharmaceutically acceptable salt thereof.

13. The method of claim 12 wherein the cancer is characterized by overexpression of Rac1 or a genomic variant thereof.

14. The method of claim 13 wherein the Rac1 genomic variant is Rac1b or Rac1 P29S.

15. The method of claim 12 wherein the cancer is selected from breast cancer, prostate cancer, ovarian cancer, melanoma, colorectal cancer, and renal cell carcinoma.

16. The method of claim 9 wherein the cancer is ER+ or HER2+ breast cancer.

17. The compound of claim 1 represented by the following structural formula: 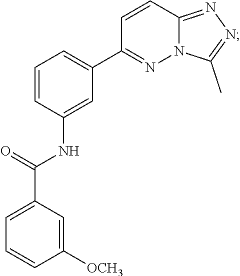 [SMILES: COc1cccc(C(=O)Nc2cccc(-c3ccc4nnc(C)n4n3)c2)c1]
or a pharmaceutically acceptable salt thereof.

18. The compound of claim 1 represented by the following structural formula: 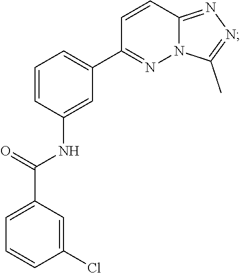 [SMILES: Cc1nnc2ccc(-c3cccc(NC(=O)c4cccc(Cl)c4)c3)nn12]
or a pharmaceutically acceptable salt thereof.

19. The compound of claim 1 represented by the following structural formula: 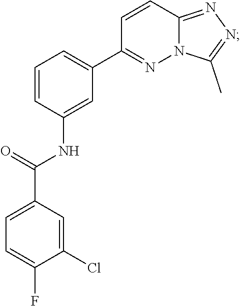 [SMILES: Cc1nnc2ccc(-c3cccc(NC(=O)c4ccc(F)c(Cl)c4)c3)nn12]
or a pharmaceutically acceptable salt thereof.

20. The compound of claim 1 represented by the following structural formula: 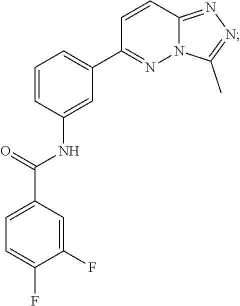 [SMILES: Cc1nnc2ccc(-c3cccc(NC(=O)c4ccc(F)c(F)c4)c3)nn12]
or a pharmaceutically acceptable salt thereof.
